(19) United States
(12) Patent Application Publication
(10) Pub. No.: US 2004/0010812 A1
(43) Pub. Date: Jan. 15, 2004

(54) Human hyaluronan receptor

(76) Inventors: Eva A. Turley, Toronto (CA); Joycelyn Entwistle, Winnipeg (CA)

Correspondence Address: FISH & NEAVE, 1251 AVENUE OF THE AMERICAS, 50TH FLOOR, NEW YORK, NY, 10020-1105, US

(21) Appl. No.: 10/205,647

(22) Filed: Jul. 23, 2002

Related U.S. Application Data
(63) Continuation of application No. 09/169,077, filed on Oct. 8, 1998, now abandoned.
Application No. 09/169,077 is a continuation of application No. PCT/CA97/00240, filed on Apr. 10, 1997.

(30) Foreign Application Priority Data
Apr. 10, 1996 (GB) 9607441.4

(51) Int. Cl. A01K 67/00; C12Q 1/68; C07H 21/04; A61K 48/00; C12P 21/02; C12N 5/06

(52) U.S. Cl. 800/8; 514/44; 435/6; 435/69.1; 435/320.1; 435/325; 530/350; 536/23.2

(57) ABSTRACT

The invention provides the genomic and cDNA sequences of human RHAMM as well as diagnostic and prognostic tests for malignancy in humans.

The present invention relates to the human hyaluronan receptor, known as the Receptor for Hyaluronic Acid Mediated Motility or RHAMM. More particularly, it relates to the genomic and cDNA sequences of the human hyaluronan receptor.

### BACKGROUND OF THE INVENTION

In the description which follows, references are made to certain literature citations which are listed at the end of the specification.

Hyaluronan is a large glycosaminoglycan that is ubiquitous in the extracellular matrix and whose synthesis has been linked to cell migration, growth and transformation. This glycosaminoglycan interacts with cell surfaces via specific protein receptors that mediate many of its biological effects.

One of these receptors is RHAMM. A RHAMM cDNA was originally cloned from a murine 3T3 fibroblast cDNA expression library (Hardwick et al., (1992) and several RHAMM isoforms were found to be encoded within the murine gene (Entwistle et al., (1995).

RHAMM acts downstream of ras in the ras transformation pathway (Hall et al., 1995). It regulates focal adhesion turnover, is required for cell locomotion and is transforming when overexpressed in murine cells (Hall et al., 1995).

### SUMMARY OF THE INVENTION

In accordance with one embodiment of the invention, an isolated nucleic acid comprises a nucleotide sequence encoding a protein selected from the group consisting of human RHAMM 1, human RHAMM 2, human RHAMM 3, human RHAMM 4 and human RHAMM 5.

In accordance with a further embodiment of the invention, an isolated nucleic acid comprises a nucleotide sequence selected from the group consisting of

(a) a nucleotide sequence of at least 10 consecutive nucleotides of Sequence ID NO:3;

(b) a nucleotide sequence of at least 15 consecutive nucleotides of Sequence ID NO:3; and

(c) a nucleotide sequence of at least 20 consecutive nucleotides of Sequence ID NO:3.

In accordance with a further embodiment of the invention, an isolated nucleic acid comprises a nucleotide encoding at least one binding domain of human RHAMM protein or a fragment or analogue thereof which retains HA binding ability.

In accordance with a further embodiment of the invention, an isolated nucleic acid comprises a nucleotide sequence of at least one exon of the nucleotide sequence of Table 1.

In accordance with a further embodiment of the invention, an isolated nucleic acid comprises a nucleotide sequence encoding the amino acid sequence of Sequence ID NO:50.

In accordance with a further embodiment, the invention provides a transgenic animal wherein a genome of the animal, or of an ancestor thereof, has been modified by insertion of at least one recobinant construct to produce a modification selected from the group consisting of

(a) insertion of a nucleotide sequence of at least one exon of the human RHAMM gene;

(b) insertion of a nucleotide sequence encoding at least one human RHAMM protein;

(c) inactivation of an endogenous RHAMM gene.

In accordance with a further embodiment, the invention provides a substantially pure protein selected from the group consisting of human RHAMM 1, human RHAMM 2, human RHAMM 3, human RHAMM 4 and human RHAMM 5.

In accordance with a further embodiment, the invention provides a substantially pure peptide comprising an amino acid sequence selected from the group consisting of

(a) at least 5 consecutive amino acid residues from the amino acid sequence of Sequence ID NO:4;

(b) at least 10 consecutive amino acid residues from the amino acid sequence of Sequence ID NO:4; and

(c) at least 15 consecutive amino acid residues from the amino acid sequence of Sequence ID NO:4.

In accordance with a further embodiment of the invention, a substantially pure peptide comprises at least one binding domain of human RHAMM.

In accordance with a further embodiment, the invention provides a substantially pure peptide having the amino acid sequence of Sequence ID NO:50.

In accordance with a further embodiment, the invention provides an antibody which selectively binds to an antigenic determinant of a human RHAMM protein.

In accordance with a further embodiment, the invention provides an antibody which selectively binds to an antigenic determinant of the peptide of Sequence ID NO:50.

In accordance with a further embodiment, the invention provides a method for identifying compounds which can selectively bind to a human RHAMM protein comprising the steps of

providing a preparation of at least one human RHAMM protein;

contacting the preparation with a candidate compound; and

detecting binding of the RHAMM protein to the candidate compound.

In accordance with a further embodiment, the invention provides a method for assessing prognosis in a mammal having a tumour, comprising obtaining a tumour sample from the mammal and determining the level of expression of RHAMM protein in the tumour sample, wherein increased expression of RHAMM protein is indicative of a poor prognosis.

In accordance with a further embodiment, the invention provides a pharmaceutical composition for preventing or treating a disorder in a human characterised by overexpression of the RHAMM gene comprising an effective amount of a nucleotide sequence selected from the group consisting of

(a) a dominant suppressor mutant of the RHAMM gene;

(b) an antisense sequence to human RHAMM cDNA; and

(c) an antisense sequence to exon 8 of the human RHAMM gene and a pharmaceutically acceptable carrier.

In accordance with a further embodiment, the invention provides a method for preventing or treating a disorder in a human characterised by overexpression of the RHAMM gene comprising administering to the mammal an effective amount of a nucleotide sequence selected from the group consisting of

(a) a dominant suppressor mutant of the RHANN gene;

(b) an antisense sequence to human RHAMM cDNA; and

(c) an antisense sequence to exon 8 of the human RHAMM gene.

In accordance with a further embodiment, the invention provides a method for inhibiting cell migration in a human comprising administering to the human an effective amount of an agent selected from the group consisting of

(a) an antibody which binds specifically to human RHAMM protein or a fragment thereof; and

(b) a peptide comprising a human RHAMM HA-binding domain.

### BRIEF DESCRIPTION OF THE DRAWINGS

Certain embodiments of the invention are described, reference being made to the accompanying drawings, wherein:

FIG. 1 shows the strategy used for cloning human RHAMM cDNA. The coding region of the human RHAMM cDNA is represented by an open rectangle, the start (ATG) and stop (TAA) codons are indicated, as are the 5′ and 3′ UTRs. The nucleotide region encoded in each clone and RT-PCR product is indicated by a single line.

FIG. 2 shows a comparison of the amino acid sequences of the HA-binding domains of mouse, rat and human RHAMM. Specifically-spaced basic amino acids corresponding to the termini of the various consensus hyaluronan-binding motifs, X¹—Aₙ—X², contained within the binding domains are underlined in the mouse sequence, amino acids 402 to 412 (Sequence ID NO:1) and amino acids 424 to 433 (Sequence ID NO:2), the numbering indicating the amino acid position of the HA-binding domains within the published amino acid sequence of mouse RHAMM2 (Hardwick et al., 1992). In the rat and human binding domains, amino acids identical to the mouse sequence are represented by dots.

FIG. 3 shows immunohistochemical staining of formalin-fixed paraffin-embedded human breast cancer tissues using an antibody to RHAMM. Sections are counterstained with methyl green. The staining intensity of tumor cells and stroma is variable. A, B, & C, General tumor staining (arrow heads) with maximum tumor staining in individual cells (arrows), D. Tumour cells and stroma both staining positively for RHAMAM, E. Tumours showing positive nuclear as well as cytoplasmic staining, and F. Tumours showing negative staining. Magnification, A and F, 400×; B,D and E, 250×; C, 650×.

FIG. 4 shows Kaplan-Meier survival curves of primary breast cancer patients subdivided according to RHAMM maximum staining.

FIG. 5 shows Kaplan-Meier survival curves for overall survival of primary breast cancer patients. The top two curves are for node negative and the bottom two curves are for node positive patients. Open symbols are for tumors with maximum-general RHAMM staining<1 unit; closed symbols are for tumors with values≧1 unit.

FIG. 6 shows Kaplan-Meier survival curves for metastasis-free survival of primary breast cancer patients. The top two curves are for node negative and the bottom two curves are for node positive patients. Open symbols are for tumors with maximum-general RHAMM staining<1 unit; closed symbols are for tumors with values≧1 unit.

FIG. 7 shows in diagrammatic form the presence or absence of exons 7 and 8 in human RHAMM isoforms 1 to 5.

### DETAILED DESCRIPTION OF THE INVENTION

The inventors have obtained the genomic sequence for human RHAMM shown in Table 1. The human RHAMM gene spans 25.4 Kilobases and comprises 17 exons.

The inventors have also obtained and sequenced the full length cDNA for human RHAMM. The cDNA, from normal human breast, has a 2175-nucleotide open reading frame (Sequence ID NO:3), which encodes a polypeptide of 725 amino acids (Sequence ID NO:4), corresponding to a molecular weight of 84 kDa.

Western analysis of the normal human breast cell line, MCF-10A, using as probe antibody R3, an antibody to murine RHAMM aa⁴²⁵⁻⁴⁴³, demonstrated three specific RHAMM protein bands of 84, 70 and 60 kDa. The major protein, which had molecular weight 70 kDa, may be generated by alternative splicing or post translational modification of the message encoding the 84 kDa protein. Also, the second ATG codon (+346, aa 116) has a perfect Kozak configuration and may be preferentially used in vivo resulting in a 70 kDa protein. Alternatively, the human RHAMM cDNA may correspond to the minor 84 kDa protein species, a possibility suggested by the observation that murine RHAMMv4 is expressed at low amounts in nontransformed cells (Entwistle et al., 1995). The above results and the presence of a stop codon in the 5′ noncoding region, in-frame with the initiation methionine, in both the human RHAMM cDNA and RT-PCR product, indicate that the cDNA is full length.

The human RHAMM protein has been found to occur in several isoforms, shown diagrammatically in FIG. 7. Similar isoforms have been identified in the mouse. The longest isoform, corresponding to the complete cDNA, is designated RHAMM 5. A shorter version of this protein, lacking the signal peptide seen in RHAMM5, is designated RHAMM 4.

Both RHAMM 4 and RHAMM 5 include exons 7 and 8. Alternatively spliced isoforms 1 and 3 lack exon 8 and exon 7 respectively. The shortest isoform, RHAMM 2, lacks both exon 7 and exon 8 and corresponds to the first described mouse RHAMM 2.

Table 2 shows a comparison of the full length human RHAMM cDNA and murine RHAMM 4 cDNA (Sequence ID NO:5), identical nucleotides being indicated by vertical broken lines, nucleotide gaps required to maintain alignment being indicated by a dash and start and stop codons being shown in bold.

Table 3 shows a comparison of the human RHAMM amino acid sequence and the murine amino acid sequence (Sequence ID NO: 6) encoded by the nucleotides of Table 2.

Identical amino acids are indicated by vertical broken lines and conservative changes are indicated by a plus sign. The two HA binding domains are shown in bold and exon 8 of the murine RHAMM 4 is underlined. Amino acid deletions, to maintain alignment, are indicated by a dash and the stop codon is indicated by an asterisk. The homology between comparable mouse and human RHAMM isoforms is 85%.

Only one of the five amino acid repeat sequences encoded in murine RHAMM cDNA (double underlined in Tables 2 and 3) are present in human RHAMM cDNA.

Alternatively spliced exon 8 has been shown to be critical to the function of RHAMM in cell motility, proliferation and transformation of murine cells (Entwistle, 1994; Hall, 1995). A review of RHAMM expression in human tissues has shown that most normal tissues contain human RHAMM 1 isoform, and do not contain detectable RHAMM 4. In contrast, tumor tissues and normal tissues responding to injury show expression of the RHAMM 4 isoform.

Alternatively spliced human exon 8 (Sequence ID NO:16) encodes the amino acid sequence VSIEKEKIDEKSETEKLLEYIEEIS (Sequence ID NO:50).

As previously described (International Patent Application WO93/21312), murine RHAMM demonstrates a consensus binding motif, X¹—Aₙ—X², wherein X¹ and X² are basic amino acid residues and Aₙ is an amino acid sequence comprising seven or eight neutral or basic amino acid residues. Several versions of this motif occur within the two murine RHAMM binding domains, at amino acids 402 to 412 and 424 to 433 of the murine RHAMM 2 amino acid sequence. As seen in FIG. 2 and Table 3, this binding motif is completely conserved in the rat and human RHAMM binding domains. For human RHAMM, the binding domains comprise the amino acid sequence KQKIKHVVKLK (Sequence ID NO:1) and KLRCQLAKKK (Sequence ID NO: 7).

Nucleic Acids

In accordance with one series of embodiments, the present invention provides isolated nucleic acids corresponding to or relating to the human RHAMM nucleic acid sequences disclosed herein.

In accordance with another series of embodiments, the present invention provides for isolating nucleic acids which include subsets of the human RHAMM sequences or their complements. Such sequences have utility as probes and PCR primers.

Expression of RHAMM Proteins

In accordance with a further embodiment, the present invention provides nucleic acids in which the coding sequence for a human RHAMM protein is operably joined to endogenous or exogenous 5′ and/or 3′ regulatory regions. For example, the complete ORF for human RHAMM protein operably joined to exogenous regulatory regions may be used for expression of the full length human RHAMM protein. The regulatory region may be selected from sequences that control the expression of genes of prokaryotic or eukaryotic cells, their viruses and combinations thereof. Such regulatory regions include for example, but are not limited to, the lac system, the trp system, the tac system and the trc system. Regulatory elements may be selected which are inducible or respressible, to allow for controlled expression of the human RHAMM gene in cells transformed with the encoding nucleic acid. Alternatively, the coding region may be operably joined with regulatory elements which provide for tissue specific expression of the human RHAMM gene in a selected tissue.

Only selected RHAMM isoform, or a selected portion thereof, may be expressed by selecting the appropriate encoding nucleotide sequence.

For protein expression, eukaryotic and prokayotic expression systems may be generated in which the selected nucleotide sequence is introduced into a plasmid or other vector which is then introduced into living cells. Prokaryotic and eukaryotic expression systems allow various important functional domains of the protein to be recovered as fusion proteins and then used for binding, structural and functional studies and also for the generation of appropriate antibodies.

Typical expression vectors contain promoters that direct the synthesis of large amounts of mRNA corresponding to the gene. They may also include sequences allowing for their autonomous replication within the host organism, sequences that encode genetic traits that allow cells containing the vectors to be selected, and sequences that increase the efficiency with which the mRNA is translated. Some vectors contain selectable markers such as neomycin resistance that permit isolation of cells by growing them under selective conditions. Stable long-term vectors may be maintained as freely replicating entities by using regulatory elements of viruses. Cell lines may also be produced which have integrated the vector into the genomic DNA and in this manner the gene product is produced on a continuous basis.

Eukaryotic expression systems permit appropriate post-translational modifications to expressed proteins. This allows for studies of the gene and gene product including determination of proper expression and post-translational modifications for biological activity, identifying regulatory elements located in the 5′ region of the gene and their role in tissue regulation of protein expression. It also permits the production of large amounts of protein for isolation and purification, the use of cells expressing the protein as a functional assay system for antibodies generated against the protein, the testing of the effectiveness of pharmacological agents or as a component of a signal transduction system, the study of the function of the normal complete protein, specific portions of the protein, or of naturally occurring polymorphisms and artificially produced mutated proteins. The DNA sequence can be altered using procedures such as restriction enzyme digestion, DNA polymerase fill-in, exonuclease deletion, terminal deoxynucleotide transferase extension, ligation of synthetic or cloned DNA sequences and site-directed sequence alteration using specific oligonucleotides together with PCR.

Once the appropriate expression vector containing a selected nucleotide sequence is constructed, it is introduced into an appropriate E.coli strain by transformation techniques including calcium phosphate transfection, DEAE-dextran transfection, electroporation, microinjection, protoplast fusion and liposome-mediated transfection.

Suitable host cells include, but are not limited to, E. coli, pseudomonas, bacillus subtillus, or other bacilli, other bacteria, yeast, fungi, insect (using baculoviral vectors for expression), mouse or other animal or human tissue cells, or cell lines such as Cos or CHO.

Suitable methods for recombinant expression of proteins are described in Sambrook et al. (1989).

Substantially Pure Proteins:

In accordance with a further embodiment, the invention provides for substantially pure preparations of human RHAMM proteins, fragments of the human RHAMM proteins and fusion proteins including human RHAMM protein fragments. The proteins, fragments and fusions have utility, as described herein, for the production of antibodies to human RHAMM protein and in diagnostic and therapeutic methods, as described herein.

The present invention provides substantially pure proteins or peptides comprising amino acid sequences which are subsequences of the complete amino acid sequence of human RHAMM protein. The invention provides substantially pure proteins or peptides comprising sequences corresponding to at least 4 to 5 consecutive amino acids of the human RHAMM amino acid sequence, preferably 6 to 10 consecutive amino acids, and more preferably at least 50 to 100 consecutive amino acids, as disclosed herein. The proteins or peptides of the invention may be isolated or purified by standard protein purification procedures including gel filtration chromatography, ion exchange chromatography, high performance liquid chromatography or a RHAMM immunoaffinity purification. For example, a protein may be expressed as a fusion protein with glutathiones-transferase (GST) and purified by affinity purification using a glutathione column. Human RHAMM may be expressed and purified, for example, as described for murine RHAMM in European Patent Application EPO 721012A2.

Antibodies

In accordance with a further embodiment, the invention provides antibodies which selectively bind human RHAMM protein or a portion or antigenic determinant thereof. Such antibodies may be prepared by conventional methods known to those skilled in the art.

A human RHAMM protein or a portion thereof for use in antibody production may be prepared by expression of a nucleotide sequence disclosed herein or a portion thereof, as described elsewhere herein.

For a short peptide, it may be necessary to prepare a fusion protein comprising the selected peptide and a carrier protein, to act as antigen.

The selected RHAMM protein or peptide or fusion protein is injected into rabbits or other appropriate laboratory animals to raise polyclonal antibodies.

Following booster injections at weekly intervals, the rabbits or other laboratory animals are bled and their serum isolated. The serum can be used directly or the polyclonal antibodies purified prior to use by various methods including affinity chromatography.

As will be understood by those skilled in the art, monoclonal antibodies may also be produced. A selected RHAMM protein or a peptide, coupled to a carrier protein if desired, is injected in Freund's adjuvant into mice. After being injected three times over a three week period, the mice spleens are removed and resuspended in phosphate buffered saline (PBS). The spleen cells serve as a source of lymphocytes, some of which are producing antibody of the appropriate specificity. These are then fused with a permanently growing myeloma partner cell, and the products of the fusion are plated into a number of tissue culture wells in the presence of a selective agent such as HAT. The wells are then screened by ELISA to identify those containing cells making binding antibody. These are then plated and after a period of growth, these wells are again screened to identify antibody-producing cells. Several cloning procedures are carried out until over 90% of the wells contain single clones which are positive for antibody production. From this procedure a stable line of clones which produce the antibody are established. The monoclonal antibody can then be purified by affinity chromatography using Protein A Sepharose, ion-exchange chromatography, as well as variations and combinations of these techniques. Truncated versions of monoclonal antibodies may also be produced by recombinant techniques in which plasmids are generated which express the desired monoclonal antibody fragment in a suitable host.

Antibodies to RHAMM or to one or more of its HA binding domains block HA binding and inhibit cell locomotion. Since RHAMM/HA interaction is involved in oncogene- and growth factor-mediated cell locomotion, antibodies to human RHAMM, or to variants or fragments thereof which retain HA binding ability, provide means for therapeutic intervention in diseases involving cell locomotion. These diseases include tumour invasion, birth defects, acute and chronic inflammatory disorders, Alzheimer's and other forms of dementia, including Parkinson's and Huntington's diseases, AIDS, diabetes, autoimmune dieases, corneal dysplasias and hypertrophies, burns, surgical incisions and adhesions, strokes and Multiple Sclerosis. Other situations involving cell locomotion, in which intervention using antibodies to RHAMM or its constituent peptides could be employed, include CNS and spinal cord regeneration, contraception and in vitro fertilisation and embryo development. Antibodies to RHAMM hve been shown to inhibit human sperm motility in vitro and also to inhibit fertilisation of hamster ova by human sperm in an in vitro system.

Suitable methods for creation of antibodies are described, for example, in Antibody Engineering: A Practical Guide, Borrebaek, Ed., W. H. Freeman and Company, New York (1992) or Antibody Engineering, 2^{nd} Edition, Borrebaek, Ed., Oxford University Press, Oxford (1995).

Transformed Cells

In accordance with a further embodiment, the present invention provides for cells or cell lines, either eukaryotic or prokaryotic, transformed or transfected with a nucleic acid of the present invention. Such cells or cell lines are useful both for preparation of human RHAMM protein or fragments thereof as described herein. They are also useful as model systems for diagnostic and therapeutic techniques.

Methods of preparing appropriate vectors containing the nucleic acids of the invention and for transforming cells using those vectors are known to those in the art and are reviewed, for example, in Sambrook et al., (1989).

Diagnostic or Prognostic Indicator in Breast Cancer

In accordance with a further embodiment tissues suspected of malignancy may be screened by determining whether or not RHAMM 5 is overexpressed, overexpression being indicative of malignancy.

In accordance with a further embodiment, the present invention provides a method of assessing the prognosis of subjects with breast cancer.

On histological examination of breast tumours, extremely high levels of RHAMM were noted to occur in individual cells or small foci of cells (maximum staining). The presence of these cells was variable but correlated with increasing general staining for RHAMM. More significantly, the presence of these unusual cells was prognostic of poor outcome (p−0.02). When maximum staining and general staining were combined as a new statistical parameter (max-general), elevated RHAMM expression significantly added to the prognostic value of nodal status and tumor size (p=0.016 and p=0.008). The involvement of RHAMM in breast carcinoma was further assessed by analyzing RHAMM mRNA level in a second patient cohort from a different geographic area. In this second study, RHAMM mRNA expression in human tissue was significantly associated with higher tumour grade as well as with combined poor parameters (high tumour grade, ER negative and lymph node positive) (p=0.0357 and p=0.0213).

Tumour size and lymph node status have been shown to be the parameters that are significant for predicting overall survival in breast cancer patients according to analyses based on a Cox proportional hazard model. There appeared to be a relationship between RHAMM overexpression generally within tumours and the appearance of single or small groups of cells that highly overexpress RHAMM. This relationship contributes to tumour progression since a combined score representing both types of staining enhanced the prognostic value of node status and metastasis free survival. It is likely that single cells expressing very high levels of RHAMM arose from a background of cells expressing high levels of this HA receptor.

An immunohistochemical study showed that combined general and maximum RHAMM protein expression was related to survival predicated by lymph nodal status, but was independent of ER/PR status and tumour grade. A second study which focused on mRNA expression yielded similar prognostic results and also a significant association with ER/PR status and with higher tumour grade was obtained. This difference might be due to the greater sensitivity of the RT-PCR technique to detect RHAMM used in the second study.

Exon 8 Peptide

The peptide (Sequence ID No:50) encoded by human exon 8 (Sequence ID NO:16) can be synthesised, and antibodies raised to it, by conventional methods, preferably after conjugating the peptide to another antigen such as keyhole limpet haemocyanin. If mice are inoculated with conjugated antigen, spleen cells can be obtained and hybridomas produced, as will be understood by those skilled in the art. Screening by conventional methods can be carried out to obtain a hybridoma producing monoclonal antibodies with maximum affinity for the exon 8 peptide. The selected antibody can be used to construct a conventional ELISA, permitting screening of human serum or human tissues for soluble RHAMM containing the peptide coded by exon 8. Comparison with standard values obtained from normal patients can be used for comparison to indicate overexpression and the presence of tumour.

Alternatively, antibodies to exon 8 could be created from phage display libraries.

Alternatively, biopsy samples of human tumours can be examined for the level of expression of exon 8 peptide by histochemical means (paraffin sections or frozen sections), to provide an indicator of likely prognosis. Histochemistry can be carried out by conventional methods, as previously described, for example, in Wang et al., 1992, using antibody to the exon 8 peptide as probe.

It has been shown that both soluble murine GST-RHAMM fusion protein inhibits cell motility and also blocks cells in G2M of the cell cycle. The effect of the soluble fusion proteins on cell motility is due to the hyaluronan binding domains and can be mimicked by peptides that encode these hyaluronan binding domains. However, the effect of the soluble protein on cell cycle block is not currently known but is contained within RHAMM2 and is likely therefore to be the repeated sequences.

By providing the cDNA sequence for human RHAMM isoforms, the inventors have provided a means of producing soluble human RHAMM protein by expression of any of the human isoforms that include RHAMM 1, 2, 3, 4 or 5 in conventional expression systems as described above. The soluble RHAMM isoforms may be used as a means of modulating the ratio of cell associated RHAMM to soluble RHAMM thereby modifying the availability of RHAMM ligands for the cell surface form of RHAMM which regulates cell locomotion and cell cycle. It is predicted that based on the murine results RHAMM 2 would be sufficient to regulate events involving cell motility and cell cycle. However, other RHAMM isoforms might be required for regulating events in tumour progression since these additional isoforms encode exon 7 and 8 (involved in tumorigenesis) unlike RHAMM 2 which does not encode these exons. These human soluble RHAMM proteins could be used clinically for wound repair, burns, reduction of inflammation following transplantation, or prevention of tumour growth and metastasis. There are significant differences in the sequence of the human vs the murine RHAMM isoforms that require the use of the human RHAMM cDNA's for production of soluble proteins so that an immune response (which can be generated against a single amino acid change) is not generated in humans negating the beneficial effects of the fusion protein.

RHAMM Transgenic Animal Models

In accordance with a further embodiment, the present invention provides for the production of transgenic, non-human animal models for the identification of the role of the RHAMM gene during embryogenesis, growth and development and to the understanding of the disease which the gene is responsible and/or related for the testing of possible therapies. In the present invention, the development of a transgenic model for the study of the relationship between RHAMM gene expression and malignancy and in particular breast cancer is particularly advantageous.

Mice are often used for transgenic animal models because they are easy to house, relatively inexpensive, and easy to breed. Transgenic animals are those which carry a transgene, that is, a cloned gene introduced and stably incorporated which is passed on to sucessive generations. In the present invention, the human RHAMM gene may be cloned and stably incorporated into the genome of an animal. Alternatively, altered portions of the gene sequence may be used such as the RHAMM sequence which does not include exon 8, the coding region thought responsible for the development of malignancy. In this manner, the specific function of alternatively spliced gene products may be investigated during animal development and initiation of malignancy in order to develop therapeutic strategies.

There are several ways in which to create a transgenic animal model carrying a certain human gene sequence. Generation of a specific alterations of the human RHAMM gene sequence is one strategy. Alterations can be accomplished by a variety of enzymatic and chemical methods used in vitro. One of the most common methods is using a specific oligonucleotide as a mutagen to generate precisely designed deletions, insertions and point mutations in a DNA sequence. Secondly, a wild type human gene and/or humanized murine gene could be inserted by homologous recombination. It is also possible to insert an altered or mutant (single or multiple) human gene as genomic or minigene constructs using wild type or mutant or artificial promoter elements. More commonly, knock-out of the endogenous murine genes may be accomplished by the insertion of artificially modified fragments of the endogenous gene by homologous recombination. In this technique, mutant alleles are introduced by homologous recombination into embryonic stem cells. The embryonic stem cells containing a knock out mutation in one allele of the gene being studied are introduced into early mouse embryos. The resultant mice are chimeras containing tissues derived from both the transplanted ES cells and host cells. The chimeric mice are mated to assess whether the mutation is incorporated into the germ line. Those chimeric mice each heterozygous for the knock-out mutation are mated to produce homozygous knock-out mice.

Gene targeting producing gene knock-outs allows one to assess in vivo function of a gene which has been altered and used to replace a normal copy. The modifications include insertion of mutant stop codons, the deletion of DNA sequences, or the inclusion of recombination elements (lox p sites) recognized by enzymes such as Cre recombinase. Cre-lox system allows for the ablation of a given gene or the ablation of a certain portion of the gene sequence.

To inactivate a gene chemical or x-ray mutagenesis of mouse gametes, followed by fertilization, can be applied. Heterozygous offspring can then be identified by Southern blotting to demonstrate loss of one allele by dosage, or failure to inherit one parental allele using RFLP markers.

To create a transgenic mouse an altered version of the human gene of interest can be inserted into a mouse germ line using standard techniques of oocyte microinjection or transfection or microinjection into stem cells. Alternatively, if it is desired to inactivate or replace the endogenous gene, homologous recombination using embryonic stem cells may be applied as described above.

For oocyte injection, one or more copies of the normal human RHAMM gene or altered human RHAMM gene sequence can be inserted into the pronucleus of a just-fertilized mouse oocyte. This oocyte is then reimplanted into a pseudo-pregnant foster mother. The liveborn mice can then be screened for integrants using analysis of tail DNA for the presence of human RHAMM gene sequences. The transgene can be either a complete genomic sequence injected as a YAC or chromosome fragment, a cDNA with either the natural promoter or a heterologous promoter, or a minigene containing all of the coding region and other elements found to be necessary for optimum expression.

Retroviral infection of early embryos can also be done to insert the altered gene. In this method, the altered gene is inserted into a retroviral vector which is used to directly infect mouse embryos during the early stages of development to generate a chimera, some of which will lead to germline transmission.

Homologous recombination using stem cells allows for the screening of gene transfer cells to identify the rare homologous recombination events. Once identified, these can be used to generate chimeras by injection of mouse blastocysts, and a proportion of the resulting mice will show germline transmission From the recombinant line. This gene targeting methodology is especially useful if inactivation of the gene is desired. For example, inactivation of the gene can be done by designing a DNA fragment which contains sequences from a exon flanking a selectable marker. Homologous recombination leads to the insertion of the marker sequences in the middle of an exon, inactivating the gene. DNA analysis of individual clones can then be used to recognize the homologous recombination events.

It is also possible to create mutations in the mouse germline by injecting oligonucleotides containing the mutation of interest and screening the resulting cells by PCR.

This embodiment of the invention has the most significant commercial value as a mouse model for breast cancer. The role of RHAMM can be idenitified during growth and development of mice to study its expression and effects on tissues with respect to malignancy. Since exon 8 has been identified to be responsible for malignancy, transgenic mice carrying this exon as well as transgenic mice having the RHAMM gene devoid of exon 8 or carrying additional copies of this exon can be made and studied with respect to malignancy and used as a model to study possible therapies including pharmaceutical intervention, gene targeting techniques, antibody therapies etc.

Antisense (AS) Therapy

The invention provides a method for reversing a transformed phenotype resulting from the expression of the RHAMM human gene sequence which includes exon 8, the exon thought responsible for transformation of cells into a malignant phenotype. Antisense based strategies can be employed to explore gene function, inhibit gene function and as a basis for therapeutic drug design. The principle is based on the hypothesis that sequence specific suppression of gene expression can be achieved by intracellular hybridization between mRNA and a complementary anti-sense species. It is possible to synthesize anti-sense strand nucleotides that bind the sense strand of RNA or DNA with a high degree of specificity. The formation of a hybrid RNA duplex may interfere with the processing/transport/translation and/or stability of a target mRNA.

Hybridization is required for an antisense effect to occur. Antisense effects have been described using a variety of approaches including the use of AS oligonucleotides, injection of AS RNA, DNA and transfection of AS RNA expression vectors.

Therapeutic antisense nucleotides can be made as oligonucleotides or expressed nucleotides. oligonucleotides are short single strands of DNA which are usually 15 to 20 nucleic acid bases long. Expressed nucleotides are made by an expression vector such as an adenoviral, retroviral or plasmid vector. The vector is administered to the cells in culture, or to a patient, whose cells then make the antisense nucleotide. Expression vectors can be designed to produce antisense RNA, which can vary in length from a few dozen bases to several thousand.

AS effects can be induced by control (sense) sequences. The extent of phenotypic changes are highly variable. Phenotypic effects induced by AS are based on changes in criteria such as biological endpoints, protein levels, protein activation measurement and target mRNA levels.

Multidrug resistance is a useful model for the study of molecular events associated with phenotypic changes due to antisense effects since the MDR phenotype can be established by expression of a single gene mdrl (MDR gene) encoding P-glycoprotein (a 170 kDa membrane glycoprotein, ATP-dependent efflux pump).

In the present invention, mammalian cells in which the RHAMM cDNA has been transfected and which express a malignant phenotype, can be additionally transfected with anti-sense RHAMM DNA sequences in order to inhibit the transciption of the gene and reverse or reduce the malignant phenotype. Alternatively, portions of the RHAMM gene can be targeted with an anti-sense RHAMM sequence specific for exon 8 which is responsible for the malignant phenotype. Expression vectors can be used as a model for anti-sense gene therapy to target the RHAMM gene including exon 8 which is expressed in malignant cells. In this manner malignant cells and tissues can be targeted while allowing healthy cells to survive. This may prove to be an effective treatment for malignancies induced by RHAMM.

Protein Therapy

Treatment of malignant disease due to overexpression of the human RHAMM gene containing exon 8 can be performed by replacing the entire translated protein with a spliced protein which does not include the exon 8 protein sequence, or by modulating the function of the entire protein sequence. Once the biological pathway of the RHAMM protein has been completely understood, it may also be possible to modify the pathophysiologic pathway (eg. a signal transduction pathway) in which the protein participates in order to correct the physiological defect.

To replace the protein with a spliced protein, or with a protein bearing a deliberate counterbalancing mutation it is necessary to obtain large amounts of pure RHAMM protein from cultured cell systems which can express the protein. Delivery of the protein to the effected tissues can then be accomplished using appropriate packaging or administering systems.

### EXAMPLES

The examples are described for the purposes of illustration and are not intended to limit the scope of the invention.

Methods of molecular genetics, protein and peptide biochemistry and immunology referred to but not explicitly described in this disclosure and examples are reported in the scientific literature and are well known to those skilled in the art.

### Example 1

Cloning and DNA Sequencing:

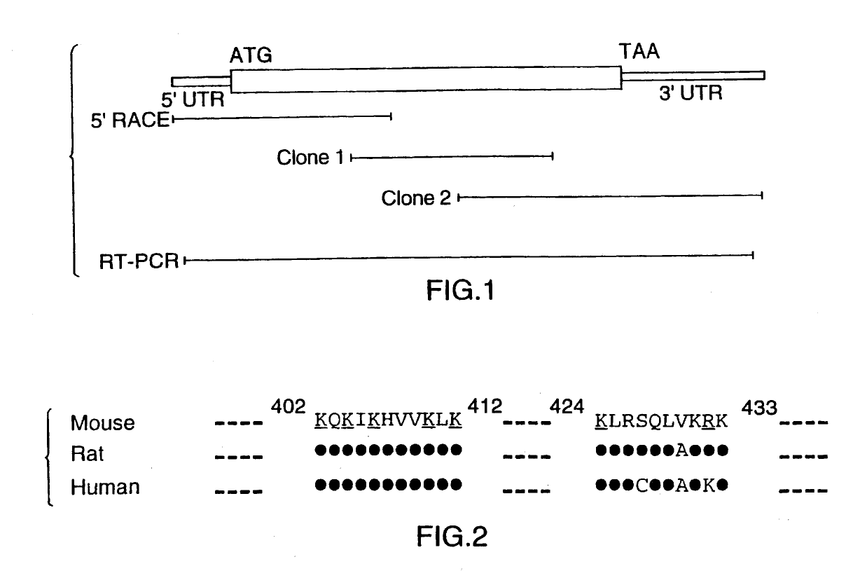

A 5′-stretch normal human breast cDNA library in lambda gt11 was obtained from Clontech (Palo Alto, Calif.) and screened using as probe the murine RHAMM 2 cDNA. Two positive clones (clones 1 & 2, FIG. 1) were PCR amplified using the 5′ and 3′ insert screening amplifiers from the λgt11 vector. The resulting 1.4 kb and 1.7 kb inserts were cloned into the PCR™ TA vector (Invitrogen, San Diego, Calif.) and sequenced by the dideoxy chain termination method using the T7 Sequencing™ kit (Pharmacia Biotech, Uppsala, Sweden). The resulting cDNA sequence was missing the amino terminal region. Using Marathon™ cDNA amplification kit (Clontech), generated from the coding region of the human cDNA clone 1, a 1.4 kb 5′ RACE fragment was obtained from mRNA from a normal human breast epithelial cell line, MCF-10A (ATCC, Rockville, Md.). This product was cloned into pCR™ TA cloning vector and sequenced as described above. The sequence obtained from these two sources was a 2.8 kb fragment and contained an ORF of 2175 nt. The strategy used for cloning this cDNA is shown in FIG. 1.

Cell Line and Culture Condition:

The normal human breast epithelial cell line, designated MCF-10A, was obtained at passage 40 from ATCC (Rockville, Md.). The cells were grown in Dulbecco's minimal essential medium (DMEM)/F-12 (1:1) medium) supplemented with 5% equine serum, 0.1 μg/ml cholera toxin, 10 μg/ml insulin (Gibco BRL, Burlington, ON), 0.5 μg/ml hydrocortisone (Sigma Chemical Co., St. Louis, Mo.) and 0.02 μg/ml epidermal growth factor (Collaborative Research, Inc., Palo Alto, Calif.) at 37° C. and 5% CO₂ in air.

Isolation of RNA from Cells:

mRNA was extracted from 90% confluent cultures of the normal breast epithelial cell line, MCF-10A, using the Micro-FastTrack™ kit following the manufacturer's instructions. Briefly, the cells were initially lysed in detergent-based buffer containing RNase/Protein Degrader, incubated at 45° C. and applied directly to Oligo (dT) cellulose for adsorption. DNA, degraded proteins, and cell debris were washed from the resin with a high salt buffer (Binding buffer). Non-polyadenylated RNAs were washed off with a low salt buffer and the PolyA⁺RNA was then eluted in the absence of salt. Purity and quantity of the RNA was assessed by reading optical densities at 260 and 280 nm.

Reverse Transcription-Polymerase Chain Reaction (RT-PCR):

To confirm that the ORF of the human RHAMM cDNA obtained from the library was full length, RT-PCR amplification using isolated RNA from a human breast epithelial cell line followed by DNA sequencing was performed. Reverse transcription was performed exactly as described in the first-strand cDNA synthesis kit (Clontech) according to manufacturer's instructions. Briefly, 1 μg messenger RNA, extracted as described above, was reverse transcribed using a 16-mer oligo dT primer and 100U MMLV reverse transcriptase at 42° C. for 60 min. The total 20 μl reaction was diluted to 100 μl by adding 80 μl of sterile water. 10 μl of the diluted cDNA template was used In each 50 μl PCR reaction using thermostable Taq and Pwo DNA polymerases (Boehringer-Mannhelm Expand™ Long Template PCR System). TaqStart antibody (Clontech), and primers 5′ GGATATCTGCAGAATTCGGCTTACT (Sequence ID NO:51) and 5′ ACAGCAACATCAATAACAACAAGA (Sequence ID NO:52) derived from the human RHAMM cDNA noncoding regions. PCR cycling parameters were denaturation at 94° C. for 1 min, denaturation at 94° C. for 45 sec, annealing at 60° C. for 45 sec and extension at 68° C. for 2 min. 35 cycles were used with a final extension time of 8 min. The PCR products were cloned into pCR™ TA cloning vector and sequenced as described above.

Western Immunoblot Analysis

The MCF-10A cells were grown in growth media and changed to defined media for 24 hours before harvest. After washing with ice cold PBS, the cells were lysed with ice cold modified RIPA lysis buffer (25 mM Tris HCl, pH 7.2, 0.1% SDS, 1% Triton-X 100, 1% sodium deoxycholate, 0.15 M NaCl, 1 mM EDTA) containing the protease inhibitors leupeptin (1 μg/ml), phenylmethyl sulfonyfluoride (PMSF, 2 mM), pepstatin A (1 μg/ml), aprotinin (0.2 TIU/ml) and 3,4-dichloroisocoumarin (200 μM) (all chemicals are from Sigma). Lysates were centrifuged at 13,000 rpm for 20 min at 4° C. (Heraeus Biofuge 13, Baxter Diagnostics Corporation, Mississauga, Ontario) following 20 min incubation on ice. Protein concentrations of the supernants were determined using the DC protein assay (Bio-Rad Laboratories, Richmond, Calif.) Five μg of total protein from each cell lysate in SDS reducing sample buffer was loaded and separated by electrophoresis on a 10% SDS-PAGE gel together with prestained molecular weight standards (Sigma). After transferring onto nitrocellulose membranes (Bio-Rad) in a buffer containing 25 mM Tris-HCl, 192 mM glycine, 20% methanol, pH 8.3, using electrophoretic transfer cells (Bio-Rad) at 100 V for 1 hour at 4° C., additional protein binding sites on the membranes were blocked with 5% defatted milk in TBST (10 nM Tris base, 150 mM NaCL, pH 7.4, with 0.1% Tween 20, Sigma). The membranes were then incubated with either the primary RHAMM antibody R3, 1:100, 1 μg/ml in defatted milk TBST) or R3.6 preincubated with murine fusion protein overnight at 4° C. on a gyratory shaker. After washing 3 times with TBST, the membranes were incubated with horseradish peroxidase-conjugated goat anti-rabbit IgG (1:5000 dilution in 1% defatted milk in TBST) for 1 hour at room temperature and washed with TBST, then TBS. Blotting was visualized by chemiluminescence (ECL) Western blotting detection system (Amersham International Plc., Amersham, UK) according to the manufacturer's instructions.

### Example 2

Materials and Methods:

Patients and Samples

The first cohort comprised archival materials from primary invasive breast carcinomas of 400 patients that had been surgically excised at the Massachusetts General Hospital from 1979 to 1982. These were used to determine the relationship of RHAMM protein overexpression with previously determined pathobiological factors and with survival. These patients continued their clinical care at Massachusetts General Hospital. The following information was obtained from the patient's clinical and medical records: age at diagnosis, location or primary tumour, time to metastasis, site of metastasis, therapeutic intervention, overall survival time, and cause of death. The median follow-up time was 10.6 years, with a minimum of one year, a maximum of 16 years, and 75% of cases having follow-up of greater than 10 years.

The second cohort comprised 98 human breast tumour specimens obtained from the NCIC-Manitoba Breast Tumour Bank. In all cases, specimens obtained for the bank have been rapidly frozen at −70° C. after surgical removal. Subsequently a portion of the frozen tissue from each case was processed to create formalin-fixed and paraffin embedded tissue blocks that were matched and oriented relative to the frozen tissue. These paraffin blocks provided tissue for high quality histological sections for pathological interpretation and assessment of the corresponding frozen tissue. Tumours were selected from the Tumour Bank database to represent a range of pathological grade (Nottingham system, score 4 to 9 corresponding to low to high grade) (Elston, 1991) and estrogen receptor status (as determined by ligand binding assay). Specific frozen tissue blocks were chosen in each case on the basis of several further criteria as assessed in immediately adjacent histological sections. These criteria included a cellular content of greater than 30% invasive tumour cells with minimal normal lobular or ductal epithelial components, good histological preservation and absence of necrosis. The majority of tumours were primary invasive ductal carcinomas.

Antibodies

The polyclonal antibodies used in this study, R3 and anti-fusion protein antibody, were raised in rabbits, R3 to a specific peptide (aa⁴²⁵⁻⁴⁴³) encoded in the murine RHAMM cDNA (Hardwick, 1992) which is conserved in human RHAMM cDNA (Table 2), and anti-fusion protein antibody to glutathione transferase (GST)-RHAMM fusion protein (Yang et al, 1993) respectively. Rabbit IgG and R3 preincubated with murine RHAMM fusion protein were used as control.

Immunohistochemistry

Routine formalin-fixed, paraffin-embedded tissues were cut into 4 micron sections and mounted on poly-lysine coated slides for assessing RHAMM expression. The Avidin-biotin-peroxidase complex method was used as previously described for CD44 staining (Yang, 1992) but with the following modifications. The slides were incubated with 1.5% goat serum in 0.01M Tris-buffered saline (TBS) for 1 hour to block non-specific binding. The primary antibody, R3 was diluted with 1.5% goat serum/TBS (1:600) and incubated on slides overnight at 4° C. Endogenous peroxidase activity was blocked by incubating the slides with 0.6% H₂O₂ in methoanol (Mallinckrodt) for 30 minutes at room temperature. The dilution of antibody was chosen by determining the dilution at which no staining was observed for reduction mammoplasties. The slides were then incubated with biotinylated goat anti-rabbit IgG (Vectastain ABC peroxidase kit, Vector Labs, Burlingame, Calif., 1:200 in 0.01M TBS) for 1 h at room temperature, following by an avidin-biotin-peroxidase complex (Vectastain, Vector labs, 1:200 in 0.01M TBS) to visualize bound antibody. Between each step, the slides were washed three times with 0.01M TBS. The peroxidase activity was developed by incubation in 0.05% DAB (3,3′-diaminobenzidine, Sigma) and 0.1% H₂O₂ in 0.05M TBS. The slides were counter-stained with methyl-green. Non-immune sera as well as antibody preabsorbed with RHAMM fusion (recombinant) protein was used as negative control.

The extent of reactivity of human breast cancer tissues to RHAMM was assessed by two independent and blinded observers without knowledge of clinical outcome. The staining intensity was scored using an arbitrary scale of 0 to 4+(0=negative, 4+=strongly positive).

Four measures of staining intensity were tested. It was not known a priori which of the four scoring measures would turn out to be significant, nor what cut-point would be useful for any of them. These four measures were: 1) general overall intensity of staining; 2) scoring of foci or isolated multiple individual cells containing the most intense staining, referred to as “maximum staining”; 3) staining with peritumour stroma; and 4) nuclear staining. The impetus for scoring “maximal scoring” came from the custom in Surgical Pathology to confer the overall diagnostic evaluation of a malignancy from the “worst” or most omnious area of a slide.

Extraction of RNA

Total RNA was extracted from one to three 20 μm frozen tumour sections as described by Hiller et al (1996) using a small scale RNA extraction protocol (Tri-Reagent, Molecular Research Center, Inc., Cincinnati, Ohio) ensuring a direct correlation between the material analyzed and histologically assessed cellular composition. The yield from tumour sections was quantitated by spectrophotometer in a 50 μl microcuvette. The average yield of total RNA per 20 μm section was 4 μg/cm² (+/−20% variation with cellularity) and this was associated with a consistent OD^{260/280}>1.8.

Reverse Transcription-Polymerase Chain Reaction (RT-PCR)

Analysis:

The expression of RHAMM was assessed by RT-PCR followed by agarose electrophoresis and ethidium bromide staining to visualize the PCR products. Amplification of actin was performed in parallel to control for reliability of reverse transcription of amplification. RHAMM isoform bands were then assessed by subjective scoring of band presence and intensity (0,0.5,1,2).

Reverse transcription was performed with 100 ng total RNA with 1 mM dNTP, 1 unit RNase inhibitor, 2.5 mM oligo d(T) primer, 50 units of MMLV reverse transcriptase and 1×MMLV buffer (Gibco BRL) in a total volume of 10 μl of 60 minutes at 37° C. Following 5 minutes incubation at 95° C., the reaction was then diluted to 40 μl and 1 μl of the cDNA (equivalent to 2.5 ng of the input RNA) was then subjected to PCR.

PCR amplifications were conducted using 1 μl of reverse transcription mixture in a volume of 50 μl, in the presence of 10 mM Tris-HCl (pH 8.3), 50 mM HCl, 1.5 mM MgCl₂, 0.2 mM dATP, 0.2 mM dCTP, 0.2 mM dGTP, 0.2 mM dTTP, 100 ng of each prime, and 2 U of Taq DNA polymerase. The primers used for RHAMM were the forward primer 5′-GCAAACACTGGATGAGCTTGA-3′ and the reverse prier 5′-TGGTCTGCTGATCTAGAAGCA-3′. PCR cycling parameters were denaturation at 94° C. for 4 min, denaturation at 94° C. for 45 sec, annealing at 60° C. for 45 sec and extension at 72° C. for 2 min. 45 cycles were used with a final extension time of 8 min. RT-PCR products were analyzed on 1% agarose gels with ethidium bromide (200 ng/ml). The 416 bp band, and in some cases additional 266 bp band were observed. These bands were cut out for sequencing. Semi-quantitative analysis of the relative amounts of RHAMM transcripts expressed was determined by comparing the expression of the RHAMM gene with that of human actin gene, the primers for which were the forward primer, 5′-ATCTGGCACCACACCTTCTACAATGAGCTGCG-3′ and the reverse primer, 5′-CGTCTACACCTAGTCGTTCGTCCTCATACTGC-3′. This resulted in a 838 bp fragment (Clontech).

DNA Sequencing

The DNA excised from the ethidium bromide stained agarose gel was purified using Prep-A-Gene DNA purification systems (Bio-Rad) according to the manufacturer's instruction and cloned into the pCR™TA vector (Invitrogen, San Diego, Calif.). it was then sequenced by the dideoxy chain termination method using the T7 Sequencing™ kit (Pharmacia Biotech, Uppsala, Sweden). A 416 bp insert corresponded to part of RHAMM 4 isoform while a 266 bp insert corresponded to RHAMM 4 minus exon 13.

Statistical Methods

Student's t-test was used for comparing the effects of RHAMM antibodies and peptides on cell locomotion and collagen gel invasion data. Kaplan-Meier survival curves were plotted for each of the scoring measures of Immunocytochemistry (Abacus, 1994). Differences between survival curves generated by using a cut-point to divide each scoring measure into a dichotomous rating (0 if below and 1 above the cut-point) were tested by the logrank (Mantel-Cox) test (Lee, 1995). A Cox proportional hazard model was used to determine the significance of multiple factors in predicting survival. Survival Tools for Statview™ was used to perform the statistical analyses (Abacus) GRAPHPAD Prism™ was used to test the difference of RHAMM mRNA expression.

RHAMM Expression in Human Breast Carcinoma

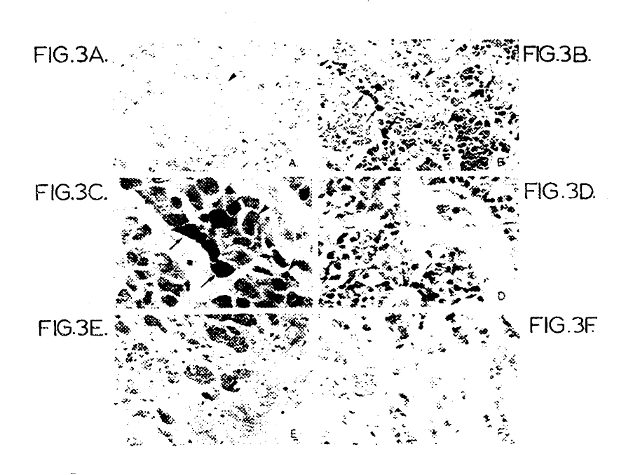

The overall expression of RHAMM protein was highly variable in a cohort of 400 human samples of breast carcinoma, ranging from most cells being negative (−) to most cells being very strongly positive (4+) (Table 4, FIG. 3). This widespread staining in the primary tumour was defined as general staining (arrow heads, FIGS. 3A-C and see FIGS. 3B-E for variability). In some tumours, RHAMM was noticably overexpressed in small foci or in multiple individual cells within the primary tumour (FIGS. 33, 3C), arrows). In these cells RHAMM was strongly expressed in both the cytoplasm and nucleus. Staining of these cells was defined as maximum staining.

For the general parameter, RHAMM was observed both within tumour cells (FIGS. 3A-D) and, in fewer cases, in the extracellular milieu, i.e. the stroma surrounding the tumour (FIG. 3E, Table 4), consistent with previous reports of the occurrence of intracellular and soluble forms in murine cells. Intracellular RHAMM appeared to be both cytoplasmic and, in some instances, nuclear (FIG. 3D). Over 80% of the 400 tumours showed no reactivity for stromal staining and nuclear staining (i.e., score 0) as noted in Table 4. It Is interesting to note that high level of general tumour staining and of maximum staining of foci and isolated cells were highly correlated (r=0.83). These correlations were significant (p<0.001). This result indicates that the appearance of small groups of cells exhibiting high expression of RHAMM (FIG. 3C) and increased staining for RHAMM are related events (i.e., compare (FIG. 3A with 3B).

RHAMM Overexpression in Cell Subsets is of Prognostic Value in Human Breast Cancer

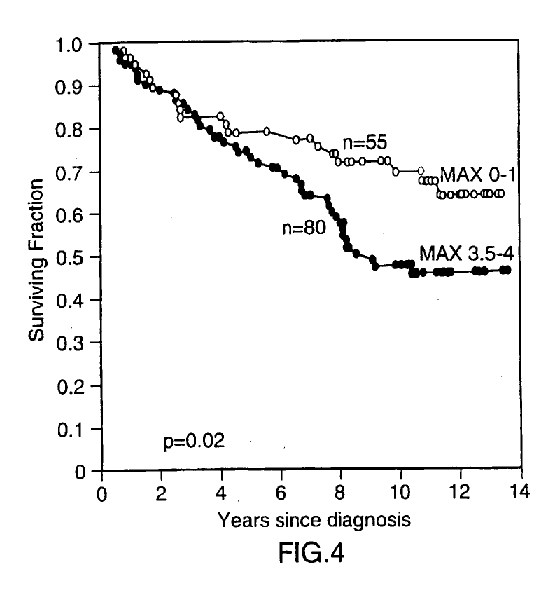

Univariate analyses of breast carcinoma tissue sections found nodal status (p<0.001) and tumour size (p=0.03) statistically significant in the first patient cohort for predicting metastasis-free and overall survival (Table 5). Neither type of RHAMM staining in this patient cohort correlated with “standard” prognostic factors (tumour size, grade, estrogen receptor status, lymph node status) (Table 5). However, RHAMM overexpression in single cells or cell subsets (maximum staining) was a prognostic factor predicting poor outcome (p=0.02) (FIG. 4).

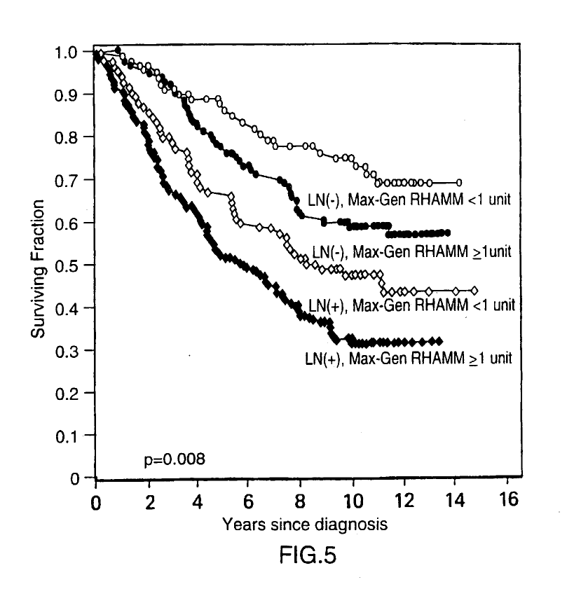

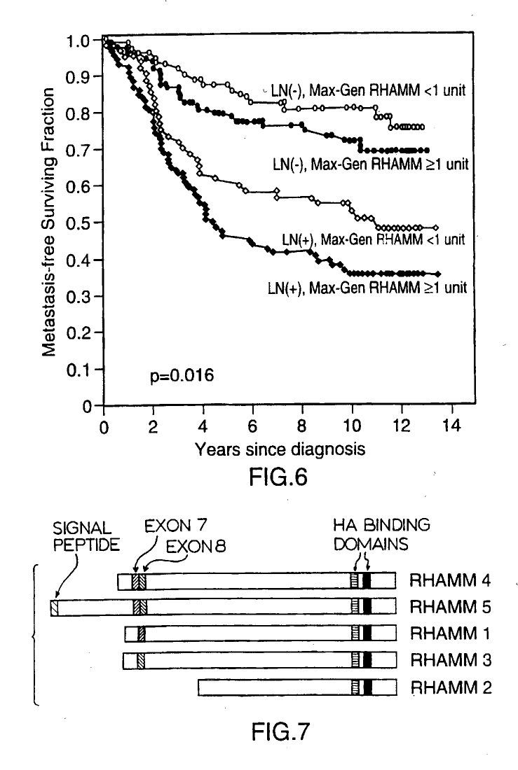

In order to assess the relationship of both maximum (Max) and general (Gen) RHAMM staining in the breast carcinoma with respect to standard prognostic factors, lymph node positive and negative patients were analyzed with a Cox proportional hazard model (Abacus, 1994; Lee, 1995), where all factors shown in Table B were included and then deleted, one at a time until only factors with p<0.05 remained in the model. This model included the number of positive lymph nodes, tumor size (classified into 3 groups: ≦2,2-5 and >5cm) as well as a combined value for general and maximal staining of RHAMM. Since maximum RHAMM staining had a negative coefficient, they were combined in a new factor which was defined as maximum-general (Max-Gen). When data were segregated according to lymph node status, the Max-Gen parameter allowed further separation of survival curves in both groups that was significant at p=0.008 for overall survival (FIG. 5) and significant at p=0.016 for metastasis-free survival (FIG. 6). These results were summarized in Table 6. The odds ratios for Max-Gen staining in this table suggest that when the Max-Gen staining difference is ≧1 unit in either group, the chance of recurrence is 1.40 times as large as then the staining difference is <1 unit. Similarly the chance of death is 1.59 times as large for those tumours with differences >1 compared to those with differences <1 unit, as seen also in FIGS. 5 and 6

RT-PCR Analysis of RHAMM Messenger RNA as Prognostic Indicator in Human Breast Carcinoma

Immunocytochemistry analysis for RHAMM protein expression in archival paraffin blocks showed a significant relationship between RHAMM overexpression and survival as well as a significant but complex association with established prognostic parameters such as lymph node status. To address this relationship further, RHAMM expression was assessed using the more sensitive technique of reverse transcription-polymerase chain reaction (RT-PCR) of mRNA extract from tissue sections from tumours of an independent cohort of 98 patients where fresh frozen tissues were available. These cases were selected specifically to provide a range of tumour grade and ER/PR status.

mRNA was detected in human breast cancer samples that corresponded to the human homologue of murine RHAMM 4. For routine analysis of RHAMM expression, RT-PCR products using primers from exon 11 and exon 14 (represented as a cDNA insert of 416 bp, see methods) were obtained (27, 31) in all tumours. A second isoform (represented as an insert of 266 bp) containing a deletion of exon 13 occurred in 29% of tumours. These results suggest that in human tumours RHAMM occurs as multiple isoforms. Protein translated from the (RHAMM 4 with exon 13 deletion) isoform would not be recognized by the antibody used for the immunohistochemical analysis as this is directed to an exon 13 epitope encoded in exon 13. Elevated expression, either of the RHAMM 4, RHAMM 4 (−9) isoforms or both isoforms combined, showed a significant association with higher tumour grade (p=0.0466, p=0.0163, p=0.0357) (Table 7). Further analysis of subsets of patients with combined parameters of poor prognosis (high grade/ER−ve/node+ve, n=12) versus patients with good prognosis (low grade/ER+ve/node−ve, n=15) showed a similar significant association of RHAMM expression with poor prognosis (p=0.0063, p=0.0085, p=0.0213) (Table 8).

### Example 3

The inventors screened human pWE15 cosmid library (Clontech) using human RHAMM 5 cDNA. Clones were mapped for restriction sites and these were lined up to match restriction sites in human RHAMM 5 cDNA. Exons were sequenced and exon/intron borders noted (Table 1).

The present invention is not limited to the features of the embodiments described herein, but includes all variations and modifications within the scope of the claims.

### REFERENCES

Abacus Concerts, Survival Tools for StatView, (1994) Berkeley: Abacus Concepts Inc.

Elston, C. W., et al., (1991), Histopathology, v. 19, pp. 403-410.

Entwistle, J., Yang, B., Wong, C., Li, Q., A., B., Mowat, M., Greenberg, A. H. and Turley E. A.: Characterization of the murine gene encoding the hyaluronan receptor RHAMM, Gene (1995), v. 163, pp. 233-238.

Hall, C., Yang, B., Yan, X., Zhang, S., Turley, M., Samuel, S., Lange, L., Wang, C., Curgen, G. D., Savani, R. C., Greenberg, A. H., and Turley, E. A., (1995), Cell, v. 82, pp. 19-28.

Hardwick, C. K., Hoare, K., Owens, R., Hohn, H. P., Hook, M., Moore, D., Cripps, V., Austen, L., Nance, M., and Turley, E. A., (1992), J. Cell. Biol., v. 117, pp. 1343-1350.

Hiller T. et al., (1996), Biotechniques, v. 21, pp. 38-44.

Lee, E. T., (1995), Statistical Methods for Survival Data Analysis, New York: John Wiley & Sons.

Sambrook et al., (1989), Molecular Cloning: A Laboratory Manual, 2^{nd} Edition, Cold Spring Harbor Laboratory Press Cold Spring Harbor, N.Y.

Wang, C., et al., (1992), Histochemistry, v. 98, pp. 105-112.

Yang, B. et al., (1993), J. Biol. Chem., v. 268, pp. 8617-8623.

Yang, B., Yang, B. L., Savani, R. C., and Turley, E. A., (1994), EMBO J., v. 13, pp. 286-296.

TABLE 1

```
EXON 1
CCGCCAGTGTGATGGATATCTGCAGAATTCGGCTTACTCACTATAGGGCTCGAGCGGCCGCC
CGGGCAGGTGTGCCAGTCACCTTCAGTTTCTGGAGCTGGCCGTCAACATGTCCTTTCCT(A)
AGGCGCCCTTGAAACGATTCAATGACCCTTCTG(INTRONXgtgcgtaagggggaaagagct
gggggggggagacgccctaacgccctttgcctctttcagctcccttcttgggaggcaagcag
gaggcgattttagggtcgggctggggctcattcagttgattgatttttctcaaatatgctct
aagcatctgttacatgccaagcactaatcaggatgctaaggataccgcagtaaacagtctcc
gcccgtgggcttacattcaggcggggaatactgtcaataaacagcggtaatggagaa.....
tttcaatccttagtaagaaagccatatattgcctgaatatatgatgtcatctcaaaactgcg
tttgctcagttgcctgtgttcctttgacccggttgatataaagggcaagatgatattgttct
tcatagagaggccttctttgtaatatcaaatggatgcaattttttacatttaaaaaaagcag
tttgttaatgacatttttacatttatattcactttattatgacatgttttaacttaagatca
                                                                                          EXON 2
taagtaacattagataatatattaatgttttctatttcctctag)GTTGTGCACCATCTCCA
GGTGCTTATGATGTTAAAACTTTAGAAGTATTGAAAGGACCAGTATCCTTTCAGAAATCACA
AAGATTTAAACAACAAAAAG(INTRONX1gtaataagatcaccaaagaacaatggttatgtg
atcttataagttttaaagttatgaataacaatatttaaagatgttatagcattttttaaaat
gtgaagctagaactatatttaaattttatttgatggatttatgaaagggtcaagtacagaat
aatgctgtcatcattacattgttatataaccaggaaaattaagcaagatacttatattgata
                              EXON 3
tgtagctt......)AATCTAAACAAAATCTTAATGTTGACAAAGATACTACCTTGCCTGCT
TCAGCTAGAAAAGTTAAGTCTTCGGAATCAA(intronxiiggtgaggagcttttatatgcc
agctggtttatcaagtgtatcatcaaaaacatctgaaagtattgtatttgattagaatgggt
taaagtgtatgaatcaaggttataactaaatctgtaaattaatgaaatgagttatcattaga
actctagcaagttttacatttctgcctaggtcattatgtttaaatgtgcccttagttcacaa
                                                                                          EXON 4
ttataatggtcttcaattctcaatcacttctatgttt......)AGAAGGAATCTCAAAAGA
ATGATAAAGATTTGAAGATATTAGAGAAAGAGATTCGTGTTCTTCTACAGGAACGTGGTGCC
CAGGACAGGCGGATCCAGGATCTGGAAACTGAGTTGGAAAAG(INTRONXX1...ttatgtt
cttaaaatgcattaagactttaagatgtatcataggtaaatatgattattcaaatagctagt
aacattagaatatctacaagcataatgtcaaaatcagagatttttccagaaactttaggggt
gattattggtagcatctccttatgttggcattctatcagtgaatcatttattatcaccttgt
ttttgtccagattcgtgttcttctacaggaacgtggtgcccaggacaggcggatccaggatc
                                        EXON 5
tggaaactgagttggaaag)ATGGAAGCAAGGCTAAATGCTGCACTAAGGGAAAAAACATCT
CTCTCTGCAAATAATGCTACACTGGAAAAACAACTTATTGAATTGACCAGGACTAATGAACT
ACTAAAATCTAAG(INTRONXXgtatctgagcctcatgataacatttacaattgaataaata
taaacacgttttttagggccgggcacggtggctcacgcctgtgttcccagcattttgggagg
ccaaggcaggcggatcacctgaggtcgggagttcgagaccagcctgaccaacatggagaaac
cctgtctctactaaaaatacaaaaattagctaggcctattggcgggcgcctgtaatctcagc
tactcgggaggctgaggcagaagaatcacttgaacccaggaggtggaggttgcagtgagc..
....taaaccagcaagtcacattaaggaaaagagggataagaacagtagactggtacagtgg
ctcatgcctgtatttccagcattttggaaggctgagggctggagaattgcttgaggccagga
gtttgagaccagcctgggcaacatatcaagaccccatctctataaacaaattgaaaaattag
ctaggcatggtggtggtgcacaccggtaatcccagctactcaggaagatgaggcaggaggat
tgattgagcccaggagtttgagattatagcgagctatgatcatgccactccactctagccgt
gacagcggagcgagacttgatctcttaaaaagaaaagaaaaaaaaattaaatcaatcagtaa
ttatggtgtaggtcaaagactgttctctctaccaaagtatattaaagtcaaaaacataaccc
cagtgataggtagaaaaatcaatatttctctattttaaatatgtcttagcagaaaatatttc
                                                            EXON 6
tgaattttttacgtgtttgttgtatttag)TTTTCTGAAAATGGTAACCAGAAGAATTTGAG
AATTCTAGCTTGGAGTTGATGAAACTTAGAAACAAAAGAGAAACAAAGATGAGG(intron
2gtgagtgctgcccttggcaggtttgctgtgtctggatctggggatcagtacaactttctca
                                                              EXON 7
tttcctaaaacaggtatctttgttgtgtag)GGTATGATGGCTAAGCAAGAAGGCATGGAGA
TGAAGCTGCAGGTCACCCAAAGGAGTCTCGAAGAGTCTCAAGGGAAAATAGCCCAACTGGAG
GGAAAACT(intron3gtaagtgagtgaatgtgaagagaattgttaagtggaagcaattct
tgatttgagtctcttcacaattattgtttactagacttaaccttctcttagtacttatctca
ttgcctccctccagttgccctatttctctttttaaactagaatgagccctaatcattctcaa
acatgttgtgctacaaagttgtatgagtgcattacttttgtacatcttctgtattattaatg
atgaggaaagatttcatgatcttatgaaagtggtcattagattgaaattgagaaacact
ggtataggaaattgtgatttatgcacaatcctagcctttgattttgagctttaatatacata
taataaaatgtgtggatagtaagtattcagtttggtgactttagcaattgtatacacctact
aaccactaccaaacaagatagaacattttcatcccttcagaaagttccttca.....#ttct
actaggtaggaagtggtatctcctttgtgattttaatttgttaccatgaatgttgaccttat
ttttatgtgcttattgaccattttatgtgcatacaacttttgcaaggtgtctattgaagtct
tttgtccatttcttgcattggacagtttggtggaggtaaacagataagtaattgaagaccag
gtagtctgggacaaaagctttatgggcacacaaaatgctatttagtatgttggatgggtggg
gaaaccaggaagaccacaaaaagaatattatttctaacacttgggatactgtaatgaaggtt
ctgtcatcataggtttttttgcagtatatattcagaaaactttctcacttaaataaaaattt
tagtcttctattttgatgtaaattgtgatttgagaaattacataaaataatagttaagagtt
agggctctgtagtcagcctgcctgatacaggagtatctggtacataagcattatgtaagatt
                                                                                                    EXON 8
attaaataacgaaactagaatgtattaacatatgcaatttttgttttag)TGTTTCAATAGA
GAAAGAAAAGATTGATGAAAAATCTGAAACAGAAAAACTCTTGGAATACATCGAAGAAATTA
G(intron4gtaatatgagcagtagctttaaattgaaccttatttttttaatactcagtcat
tttcatcatttttctgttattttccctgtgcctaaatagatgtgctttttaagataatttgt
                      EXON 9
tttaatgcag)TTGTGCTTCAGATCAAGTGGAAAAATACAAGCTAGATATTGCCCAGTTAGA
AGAAAATTTGAAAGAGAAGAATGATGAAATTTTAAGCCTTAAGCAGTCTCTTGAGGAAAATA
TTGTTATATTATCTAAACAAGTAGAAGATCTAAATGTGAAATGTCAGCTGCTTGAAAAAGAA
AAAG(intron5gtattacagtgtttatagttactttgtttagataagtgttacatacaaca
tttaggaaaaatactactatgctaaaacaaccttttaaatataattagctatactaacattt
taaatataattagctatatagctatacaacagcaaaaacctgtactgcattttagaatattt
tactcttataatgtttgttttctgtttatttcaatacagcatattacctgtcttgattgaaa
tatatacagtcatataattcttgactttccactaggtagctgtgtaacaatcagtagataac
acagaacaagatttgtgggttttattatttagcacatagtatatattacatggagtaatgat
acaaagttcacagttttgttttcttctttggaaataccatgctaaaagcagtgtaatggaat
attatgggagtccaggtttctcagtcttaatgttcttatctaattccagtattcttgatgtt
                              EXON 10
ttgagttttctag)AAGACCATGTCAACAGGAATAGAGAACACAACGAAAATCTAAATGCAG
AGATGCAAAACTTAAAACAGAAGTTTATTCTTGAACAACAGGAACATGAAAAGCTTCAACAA
AAAGAATTACAAATTGATTCACTTCTGCAACAAGAGAAA(intron6gtaatttaccaccat
atttttttaaactgttcattttgtgtcatacatttccctatgtctctgaacacctttaaatt
gtgtatatcctttgatctaccaattctatctttagagtcttatcctgaggacataatcatgg
atatgctgaggatttagctacgtattttcactacatgttcacctagggttatgaataatgtg
ggaaatgacaacagatacaaaatagggaatttttaaaaaattttctggctcattcttgtgtt
atttaggctatataaacattacacttaccttg......taattttatgtaatatggtgtgaa
aaataatgttaatatcaaagccagttgtaaaacagatatatatatataaaaatataatttta
gattaagaagtttctgcatgtgcgttgcatagaaaaaagcctaagatgatatttgccacaat
gttaacaaggtataggaaataatctatgaaaacaaatatgctatttctatattgttttaagt
ttccttgaatctgtggaatttaggtttcatccttctttatctgtacttttttttgtctccta
                                                                                                              EXON 11
gtacaacctcacaatgccattccaaattattttggtggttttctgtttggatatag)GAATT
ATCTTCGAGTCTTCATCAGAAGCTCTGTTCTTTTCAAGAGGAAATGGTTAAAGAGAAGAATC
TGTTTGAGGAAGAATTAAAGCAAACACTGGATGAGCTTGATAAATTACAGCAAAAGGAGGAA
CAAGCTGAAAGGCTGGTCAAGCAATTGGAAGAGGAAGCAAAATCTAGAGCTGAAGAATTAAA
ACTCCTAGAAGAAAAGCTGAAAGG(intron7gtttgtattaataggatctcatgttttatt
atgacttcagatgtatttattttgagtactttttttagtattctcttatcaatcatgtgagc
gtgttaggttggattatttt......ttatacctactaccttcttcacccaaatttttaaag
taaaataagcaggaaagataagttgaagctagtagaaaaatgcattaaaaaacatgctttcg
aggtaagtcataaattaggatctgagctatttagcaggtaatgcagtggtgaagatatgagc
tatatgattcacagtttcaaaggtaaatactattttctttcttagggtagtaattgtaggtg
                                                                      EXON 12
gcattttatctttcaattatttctttttcttag)GAAGGAGGCTGAACTGGAGAAAAGTAGT
GCTGCTCATACCCAGGCCACCCTGCTTTTGCAGGAAAAGTATGACAGTATGGTGCAAAGCCT
TGAAGATGTTACTGCTCAATTTGAAAG(intron8gtatttttcttgggagcctgcactctt
aaatatgatgtgtgcagaaaggggtgtttaccccaggaaatatgtgagcaaagcagtcacac
aaaggatgattcatactagtttaaattccataatcaccaaccgtaagtgggcatttagcatt
atctggtaatcttattgtatttatataattccctttataatttatagaaattcccc.....t
ttttttttctttgaatacacagcagatgccatgtaaactcattagtacttgcctcagaacac
tgaattcttacctgtgttaaatgcatgaatacattaaaaactttttagttttacttagaagt
atataaagtgtcccctaatcagttatgattgtcatacgcaatagttagaaaactactttgac
                                          EXON 13
ttttttttctttttaataag)CTATAAAGCGTTAACAGCCAGTGAGATAGAAGATCTTAAGC
TGGAGAACTCATCATTACAGGAAAAAGCGGCCAAGGCTGGGAAAAATGCAGAGGATGTTCAG
CATCAGATTTTGGCAACTGAGAGCTCAAATCAAGAATATGTAAG(intron9gtatatagag
caaataatggccttagaaccattaagacaatttaatgttgaaagccagctagtaactgtccc
ttggcttgcttttggccatcttatactgcaaattaagaatttactcagttaaaaaatgacac
ttcttgaagagttccttgaggtttaaagaaaaaaaaaggaaaaattaatgaaagtggctata
                                                                        EXON 14
aaatgtttagtgacctcttctctctcaaaccaaag)GATGCTTCTAGATCTGCAGACCAAGT
CAGCACTAAAGGAAACAGAAATTAAAGAAATCACAGTTTCTTTTCTTCAAAAAATAACTGAT
TTGCAGAACCAACTCAAGCAACAGGAGGAAGACTTTAGAAAACAGCTGGAAGATGAAGAAGG
AAG(intron10gtaatctatgattcgaacctgagtgccttgttaactcagttacgtga
                                                                                            EXON 15
ttttttaaataactatgtttttctcaatttaattcttccatgcag)AAAAGCTGAAAAAGAA
AATACAACAGCAGAATTAACTGAAGAAATTAACAAGTGGCGTCTCCTCTATGAAGAACTATA
TAATAAAACAAAACCTTTTCAG(intron11gtttgtcagttaggagtaaacttacttgtgt
ttattttagggactctttgttccctattatagtgaggacagtgactcgggttttctgcaaga
tcattttgctctgcacttacagtgccaatttagctcactattaaaggtttatacattttatt
aaattatgcataattttttcccacattattgaagtataattgacaaatttaattgacataat
ttttcaatggacctttgtggttttaaaaaaaa......ctcatagagaatctatggagagcc
ctgagaatatgtgaacataccttgttttcatttgtgtttttaattttctttagtgtttatgg
tttatatgaaactagtaagatcaaactgttttaagtcttaactttatttaaaaaatcttttt
        EXON 16
cag)CTACAACTAGATGCTTTTGAAGTAGAAAAACAGGCATTGTTGAATGAACATGGTGCAG
CTCAGGAACAGCTAAATAAAATAAGAGATTCATATGCTAAATTATTGGGTCATCAGAATTTG
AAACAAAAAATCAAGCATGTTGTGAAGTTGAAAGATGAAAATAGCCAACTCAAATCG(intr
on12gtttgtaaaatgacttttcattttattaaagatattggagtgggggttattctaacta
taatacttaaataaaatgaatatctttggtatcagaaaaaaataactgtttatagaggaaaa
ttgagctgtgatttagtggatttattttagagtgttgaccagatgggcattcaatgttctaa
agttttctagctaccgtcttaatatatattgaaaattacttgagtaaatttgatgaattcat
                                                                            EXON 17
taagctttacatatctatttccatttgcaaa......)GAAGTATCAAAACTCCGCTGTCAG
CTTGCTAAAAAAAAACAAAGTGAGACAAAACTTCAAGAGGAATTGAATAAAGTTCTAGGTAT
CAAACACTTTGATCCTTCAAAGGCTTTTCATCATGAAAGTAAAGAAAATTTTGCCCTGAAGA
CCCCATTAAAAGAAGGCAATACAAACTGTTACCGAGCTCCTATGGAGTGTCAAGAATCATGG
AAGTAAACATCTGAGAAACCTGTTGAAGATTATTTCATTCGTCTTGTTGTTATTGATGTTGC
TGTTATTATATTTGACATGGGTATTTTATAATGTTGTATTTAATTTTAACTGCCAATCCTTA
AATATGTGAAAGGAACATTTTTTACCAAAGTGTCTTTTGACATTTTATTTTTTCTTGCAAAT
ACCTCCTCCCTAATGCTCACCTTTATCACCTCATTCTGAACCCTTTGCTGGCTTTCCAGCTT
AGAATGCATCTCATCAACTTAAAAGTCAGTATCATATTATTATCCTCCTGTTCTGAAACCTT
AGTTTCAAGAGTCTAAACCCCAGATTCTTCAGCTTGATCCTGGAGGTCTTTTCTAGTCTGAG
CTTCTTTAGCTAGGCTAAAACACCTTGGCTTGTTATTGCCTCTACTTTGATTCTGATAATGC
TCACTTGGTCCTACCTATTATCCTTCTACTTGTCCAGTTCAATAAGAAATAAGGACAAGCCT
AACTTCATAGTAACCTCTCTATTTTAATCAGTTGTTTAATAATTTACAGGTTCTTAGGCTCC
ATCCTGTTTGTATGAAATTATAATCTGTGGATTGGCCTTTAAGCCTGCATTCTTAACAAACT
CTTCAGTTAATTCTTAGATACACTAAAAATCTGAAGAAACTCTACATGTAACTATTTCTTCA
GAGTTTGTCATATACTGCTTGTCATCTGCATGTCTACTCAGCATTTGATTAACATTTGTGTA
ATAAGAAATAAAATTACACAGTAAGTCATTTAACCAAAAAAAAAAAAAAAAAAAAAAAAAAA
AAAAAAAAAAAAAAAAAAAAAAAA(......ggtctgtaggaaaaacgactattgattgggt
tagcgtcctaatcgagtatgtggttctgtggctgcaacacagatgtccacagtgacaaggac
atgaacacctggatgaacgcgtctgtcaagtctgggtgggctgcatcagtgcctttgcctgt
cctgtctcttgcctaagccctcctggttctgactgctcctgcctgggtccctccttcacctg
aactctgcaggctgcacagacatgctttctgtatctgtggcccttcattgtccctttccgtg
tca......
```

TABLE 2

| | | |
|---|---|---|
| Human | −109 | CCGCCAGTGTGATGGATATCTGCAGAATTCGGCTTACTCACTATAGGGCTCGAGCGGCCGCCCGGGCAGGTGTGC |
| | −3 | CAGTCACCTTCAGTTTCTGGAGCTGGCCGTCAACATGTCCTTTCCTAAGGCGCCCTTGAAACGATTCAATGACCC |
| | 42 | TTCTGGTTGTGCACCATCTCCAGGTGCTTATGATGTTAAAACTTTAGAAGTATTGAAAGGACCAGTATCCTTTCA |
| | 117 | GAAATCACAAAGATTTAAACAACAAAAAGAATCTAAACAAAATCTTAATGTTGACAAAGATACTACCTTGCCTGC |
| | 192 | TTCAGCTAGAAAAGTTAAGTCTTCGGAATCAAAGAAGGAATCTCAAAAGAATGATAAAGATTTGAAGATATTAGA |
| | 267 | GAAAGAGATTCGTGTTCTTCTACAGGAACGTGGTGCCCAGGACAGGCGGATCCAGGATCTGGAAACTGAGTTGGA |
| | 342 | AAAGATGGAAGCAAGGCTAAATGCTGCACTAAGGGAAAAAACATCTCTCTCTGCAAATAATGCTACACTGGAA |
| Human | 415 | AAACAACTTATTGAATTGACCAGGACTAATGAACTACTAAAATCTAAGTTTTCTGAAAATGGTAACCAGAAGAAT |
| | | \| \| \| \| \| \| \|\| \| \|\| \|\| \|\|\|\|\|\|\|\|\|\|\| \|\|\|\|\| \|\|\|\| \|\|\|\|\|\| |
| Mouse | −75 | AGGCTAAAGGAGGCAGAATAGATATCTGAGTTCTTATGTTTATTGTAGTTTTCTGAAGATGGTCACCAAAAGAAT |
| Human | 490 | TTGAGAATTCTAAGCTTGGAGTTGATGAAACTTAGAAACAAAAGAGAAACAAAGATGAGGGGTATGATGGCTAAG |
| | | \|\|\|\|\| \|\|\|\|\|\|\| \|\|\|\| \|\|\|\|\|\|\|\|\|\|\| \|\|\|\|\| \|\| \|\|\|\|\| \|\|\|\|\|\|\|\|\|\|\|\| \|\|\|\|\|\|\|\|\| \|\| |
| Mouse | | ATGAGAGCTCTAAGCCTGGAATTGATGAAACTCAGAAATAAGAGAGAGACAAAGATGAGGAGTATGATGGTCAAA |
| Human | 565 | CAAGAAGGCATGGAGATGAAGCTGCAGGTCACCCAAAGGAGTCTCGAAGAGTCTCAAGGGAAAATAGCCCAACTG |
| | | \|\| \|\|\|\|\|\|\|\|\|\|\|\| \|\|\|\|\|\|\|\|\|\|\|\| \|\|\| \|\| \| \| \|\|\| \|\|\|\|\|\| \| \|\| \|\|\|\|\|\|\| \|\|\| \|\|\| |
| Mouse | 76 | CAGGAAGGCATGGAGCTGAAGCTGCAGGCCACTCAGAAGGACCTCACGGAGTCTAAGGGAAAAATAGTCCAGCTG |
| Human | 640 | GAGGGAAAACTTGTTTCAATAGAGAAAGAAAACATTGATGAAAAATCTGAAACAGAAAAACTCTTGGAATACATC |
| | | \|\|\|\|\|\|\|\| \|\|\|\|\|\|\|\|\|\|\|\|\|\|\|\|\|\|\|\|\|\|\|\|\|\| \|\|\|\|\|\|\|\|\|\| \|\|\|\|\|\|\|\|\|\|\|\|\|\|\|\|\| \|\|\|\|\|\|\|\|\|\| |
| Mouse | 151 | GAGGGAAAGGTTGTTTCAATAGAGAAAGAAAAGATCGATGAAAAATGTGAAACAGAAAAACTCTTAGAATACATC |
| Human | 715 | GAAGAAATTAGTTGTGCTTCAGATCAAGTGGAAAAATACAAGCTAGATATTGCCCAGTTAGAAGAAAATTTGAAA |
| | | \|\|\|\|\|\|\|\|\|\| \|\|\|\|\| \|\| \|\|\|\|\|\|\|\|\|\|\|\|\|\|\|\| \|\|\| \|\|\|\|\|\|\|\|\|\|\|\|\|\|\|\|\|\|\|\|\|\|\| \|\|\|\|\|\|\|\| |
| Mouse | 226 | CAAGAAATTAGCTGTGCATCTGATCAAGTGGAAAAATGCAAAGTAGATATTGCCCAGTTAGAAGAAGATTTGAAA |
| Human | 790 | GAGAAGAATGATGAAATTTTAAGCCTTAAGCAGTCTCTTGAGGAAAATATTGTTATATTATCTAAACAAGTAGAA |
| | | \|\|\|\|\|\| \|\| \|\|\| \|\|\|\|\|\|\|\| \|\|\|\|\|\|\|\|\|\|\|\|\|\|\|\|\|\|\|\|\|\|\| \|\|\| \| \|\|\| \|\|\|\|\| \|\|\| \|\|\|\|\| |
| Mouse | 301 | GAGAAGGATCGTGAGATTTTAAGTCTTAAGCAGTCTCTTGAGGAAAACATT---ACATTTTCTAAGCAAATAGAA |
| Human | 875 | GATCTAAATGTGAAATGTCAGCTGCTTGAAAAAGAAAAAGAAGACCATGTCAACAGGAATAGAGAACACAACGAA |
| | | \|\| \|\| \| \|\|\| \|\|\|\|\| \|\|\|\|\| \|\|\|\|\|\|\| \|\|\|\|\| \|\|\| \|\|\| \|\|\|\|\| \|\| \| \|\|\|\|\|\|\|\| \|\|\| |
| Mouse | 373 | GACCTGACTGTTAAATGCCAGCTACTTGAAACAGAAAGAGACAACCTTGTCAGCAAGGATAGAGAAAGGGCTGAA |
| Human | 940 | AATCTAAATGCAGAGATGCAAAACTTAAAACAGAAGTTTATTCTTGAACAACAGGAACATGAAAAGCTTCAACAA |
| | | \| \|\|\| \| \|\|\| \|\|\|\|\|\|\|\|\|\| \| \| \| \| \|\|\| \| \| \|\|\| \|\|\| \| \|\|\| \|\|\|\|\|\|\|\|\|\| \|\|\|\|\|\| |
| Mouse | 448 | ACTCTCAGTGCTGAGATGCAGATCCTGACAGAGAGGCTGGCTCTGGAAAGGCAAGAATATGAAAAGCTGCAACAA |
| Human | 1015 | AAAGAATTACAAATTGATTCACTTCTGCAACAAGAGAAAGAATTATCTTCGAGTCTTCATCAGAAGCTCTGTTCT |
| | | \|\|\|\|\|\|\|\| \|\|\|\| \| \|\|\|\|\|\|\|\|\|\|\| \|\|\|\|\|\|\|\| \|\|\| \| \|\|\| \| \|\|\|\| \|\| \|\|\| \|\|\|\|\|\|\| \|\|\| |
| | 523 | AAAGAATTGCAAAGCCAGTCACTTCTGCAGCAAGAGAAGGAACTGTCTGCTCGTCTGCAGCAGCAGCTCTGCTCT |
| Human | 1090 | TTTCAAGAGGAAATGGTTAAAGAGAAGAATCTGTTTGAGGAAGAATTAAAGCAAACACTGGATGAGCTTGATAAA |
| | | \|\| \|\|\|\|\|\|\|\|\|\|\|\| \|\|\|\|\|\|\|\| \| \|\|\| \| \|\|\|\|\| \|\|\|\|\|\| \|\|\|\| \|\|\|\| \| \|\|\| |
| Mouse | 598 | TTCCAAGAGGAAATGACTTCTGAGAAGAACGTCTTTAAAGAAGAGCTAAAGCTCGCCCTGGCTGAGTTGGATGCG |
| Human | 1165 | TTACAGCAAAAGGAGGAACAAGCTGAAAGGCTGGTCAAGCAATTGGAAGAGGAAGCAAAATCTAGAGCTGAAGAA |
| | | \| \|\|\|\|\| \|\|\|\|\|\|\|\| \|\| \|\|\|\|\|\|\|\|\|\|\|\| \|\| \|\| \|\|\|\|\|\|\|\|\|\|\| \|\| \|\| \| \|\| \|\|\| \|\| |
| Mouse | 673 | GTCCAGCAGAAGGAGGAGCAGAGTGAAAGGCTGGTTAAACAGCTGGAAGAGGAAAGGAAGTCAACTGCAGAACAA |
| Human | 1240 | TTAAAACTCCTAGAAGAAAAGCTGAAAGGGAAGGAGGCTGAACTGGAGAAAAGTAGTGCTGCTCATACCCAGGCC |
| | | \| \| \| \|\| \|\| \| \|\|\|\|\| \|\| \|\|\| \|\| \| \|\|\|\|\|\|\|\|\|\|\|\|\| \|\| \|\|\|\|\|\|\|\|\| \|\|\|\| \|\|\| |
| Mouse | 748 | CTGACGCGGCTGGACAACCTGCTGAGAGAGAAAGAAGTTGAACTGGAGAAACATATTGCTGCTCACGCCCAAGCC |
| Human | 1315 | ACCCTGCT------------------------------------------------------------------- |
| | | \| \| \|\| \| |
| Mouse | 823 | ATCTTGATTGCACAAGAGAAGTATAATGACACAGCACAGAGTCTGAGGGACGTCACTGCTCAGTTGGAAAGTGTG |
| Human | | --------------------------------------------------------------------------- |
| Mouse | 898 | CAAGAGAAGTATAATGACACAGCACAGAGTCTGAGGGACGTCACTGCTCAGTTGGAAAGTGAGCAAGAGAAGTAC |
| Human | | --------------------------------------------------------------------------- |
| Mouse | 973 | AATGACACAGCACAGAGTCTGAGGGACGTCACTGCTCAGTTGGAAAGTGAGCAAGAGAAGTACAATGACACAGCA |
| Human | 1323 | -----------------------------------TTTGCAGGAAAAGTATGACAGTATGGTGCAAAGCCTTGAA |
| | | \| \|\|\|\| \|\| \|\|\|\|\| \| \| \| \|\| \|\| \|\| |
| Mouse | | CAGAGTCTGAGGGACGTCACTGCTCAGTTGGAAAGTGTGCAAGAGAAGTACAATGACACAGCACAGAGTCTGAGG |
| Human | 1363 | GATGTTACTGCTCAATTTGAAAGCTATAAAGCGTTAACAGCCAGTGAGATAGAAGATCTTAAGCTGGAGAACTCA |
| | | \|\| \|\| \|\|\|\|\|\| \|\| \|\|\|\|\|\|\|\|\|\|\| \| \|\|\|\| \|\| \|\|\|\|\|\|\|\| \|\|\|\|\| \|\|\|\|\|\|\|\| |
| Mouse | 1123 | GACGTCAGTGCTGAGTTGGAAAGCTATAAGTCATCAACACTTAAAGAAATAGAAGATCTTAAACTGGAGAATTTG |
| Human | 1438 | TCATTACAGGAAAAAGCGGCCAAGGCTGGGAAAAATGCAGAGGATGTTCAGCATCAGATTTTGGCAACTGAGAGC |
| | | \|\|\|\| \|\|\|\|\|\|\| \|\| \| \|\|\|\|\| \|\|\|\| \|\| \|\|\| \|\|\|\|\|\|\|\| \|\| \|\|\|\|\| \|\|\| \|\| \|\|\|\|\|\|\|\| |
| Mouse | 1198 | ACTCTACAAGAAAAAGTAGCTATGGCTGAAAAAAGTGTAGAAGATGTTCAACAGCAGATATTGACAGCTGAGAGC |
| Human | 1513 | TCAAATCAAGAATATGTAAGGATGCTTCTAGATCTGCAGACCAAGTCAGCACTAAAGGAAACAGAAATTAAAGAA |
| | | \|\|\|\|\|\|\|\|\|\|\|\|\|\|\| \|\|\|\|\|\|\| \|\|\| \|\|\|\|\|\| \|\| \|\|\| \| \|\|\|\| \|\|\|\| \|\|\| \|\|\|\|\|\|\|\|\|\|\|\|\| |
| Mouse | 1273 | ACAAATCAAGAATATGCAAGGATGGTTCAAGATTTGCAGAACAGATCAACCTTAAAAGAAGAAGAAATTAAAGAA |
| Human | 1588 | ATCACAGTTTCTTTTCTTCAAAAAATAACTGATTTGCAGAACCAACTCAAGCAACAGGAGGAAGACTTTAGAAAA |
| | | \|\|\|\|\|\| \|\|\| \|\|\|\|\|\| \| \|\|\|\|\|\|\|\|\|\|\|\|\|\|\| \| \|\| \|\|\|\|\|\|\| \|\|\|\|\| \|\| \|\|\|\|\|\|\|\|\|\|\| \|\| |
| | | ATCACATCTTCATTTCTTGAGAAAATAACTGATTTGAAAAATCAACTCAGACAACAAGATGAAGACTTTAGGAAG |
| Human | 1663 | CAGCTGGAAGATGAAGAAGGAAGAAAAGCTGAAAAAGAAAATACAACAGCAGAATTAACTGAAGAAATTAACAAG |
| | | \|\|\|\|\|\|\|\|\|\|\| \|\|\| \| \|\|\|\|\| \|\|\| \|\| \|\|\|\|\|\|\|\|\| \|\| \|\|\|\|\|\|\|\|\|\| \|\|\|\|\|\|\|\| \|\| |
| Mouse | 1423 | CAGCTGGAAGAGAAAGGAAAAAGAACAGCAGAGAAAGAAAATGTAATGACAGAATTAACCATGGAAATTAATAAA |
| Human | 1738 | TGGCGTCTCCTCTATGAAGAACTATATAATAAAACAAAACCTTTTCAGCTACAACTAGATGCTTTTGAAGTAGAA |
| | | \|\|\|\|\|\|\|\|\|\|\| \|\|\|\|\|\|\|\|\|\|\|\|\|\|\| \| \|\|\|\|\| \|\|\|\|\|\|\|\|\|\|\|\|\| \|\|\|\|\|\| \|\|\|\|\| \|\|\|\|\|\|\| \|\| |
| | | TGGCGTCTCCTATATGAAGAACTATATGAAAAAACTAAACCTTTTCAGCAACAACTGGATGCCTTTGAAGCCGAG |
| Human | 1813 | AAACAGGCATTGTTGAATGAACATGGTGCAGCTCAGGAACAGCTAAATAAAATAAGAGATTCATATGCTAAATTA |
| | | \|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\| \|\|\|\|\| \|\| \|\|\|\|\| \|\| |
| Mouse | 1573 | AAACAGGCATTGTTGAATGAACATGGTGCAACTCAGGAGCAGCTAAATAAAATCAGAGACTCCTATGCACAGCTA |
| Human | 1888 | TTGGGTCATCAGAATTTGAAACAAAAAATCAAGCATGTTGTGAAGTTGAAAGATGAAAATAGCCAACTCAAATCG |
| | | \| \|\|\|\|\| \|\|\|\|\| \| \|\| \|\|\|\|\|\|\|\|\|\|\| \|\|\|\|\|\|\|\|\|\|\| \|\|\|\|\|\|\|\|\|\|\|\|\|\|\|\|\|\|\|\|\|\|\|\|\|\|\|\|\|\| |
| Mouse | 1648 | CTTGGTCACCAGAACCTAAAGCAAAAAATCAAACATGTTGTGAAATTGAAAGATGAAAATAGCCAACTCAAATCG |
| Human | 1963 | GAAGTATCAAAACTCCGCTGTCAGCTTGCTAAAAAAAAACAAAGTGAGACAAAACTTCAAGAGGAATTGAATAAA |
| | | GAGGTGTCAAAACTCCGATCTCAGCTTGTTAAAAGGAAACAAAAATGAGCTCAGACTTCAGGGAGAATTAGATAAA |
| Human | 2038 | GTTCTAGGTATCAAACACTTTGATCCTTCAAAGGCTTTTCATCATGAAAGTAAAGAAAATTTTGCCCTGAAGACC |
| | | \| \|\|\| \|\| \|\|\|\| \|\|\|\|\|\|\|\|\| \|\|\|\|\| \|\|\|\|\|\|\|\|\| \|\|\|\|\| \| \|\|\| \|\| \|\|\|\|\|\| \|\| |
| Mouse | 1798 | GCTCTGGGCATCAGACACTTTGACCCTTCCAAGGCTTTTTGTCATGCATCTAAGGAGAATTTT---------ACT |
| Human | 2113 | CCATTAAAAGAAGGCAATACAAACTGTTACCGAGCTCCTATGGAGTGTCAAGAATCATGGAAGTAAACATCTGAG |
| | | \|\|\|\|\|\|\|\|\|\|\|\|\|\|\|\|\| \|\|\|\|\|\|\| \| \| \|\|\| \|\| \|\|\| \| \|\|\|\|\|\|\|\|\|\|\|\|\|\|\|\|\|\| \|\| \|\|\|\|\| |
| Mouse | 1873 | CCATTAAAAGAAGGCAACCCAAACTGCTGCTGAGTTCAGATGCAACTTCAAGAATCATGGAAGTATACGTCTGAA |
| Human | 2188 | AAACCTGTTGAAGATTATTTCATTCGTCTTGTTGTTATTGATGTTGCTGTTATTATATTTGACATGGGTATTTTA |
| | | \|\| \|\|\|\|\|\|\|\|\|\|\|\|\|\|\| \|\|\| \| \| \| \| \| \| \| \|\|\|\| \|\|\| \|\| \|\| \| \| |
| Mouse | 1948 | ATACTTGTTGAAGATTATTTTCTTCATTGTTCTTGTTATAGTATATAATGPATTTAATTTCTACTGCGTAGTCTT |
| Human | 2023 | TAATGTTGTATTTAATTTTAACTGCCAATCCTTAAATATGTGAAAGGAACATTTTTTTTCCAAGTGTCTTTTGAC |
| | | \| \| \| \| \| \| |
| Mouse | 2023 | AGGTATATGAAACGGTAATTCAGCATTTGTTCTCT---------------------------------------- |
| Human | 2338 | ATTTTATTTTTTCTTGCAAATACCTCCTCCCTAATGCTCACCTTTATCACCTCATTCTGAACCCTTTCGCTGGCT |
| Human | 2413 | ATTTTATTTTTTCTTGCAAATACCTCCTCCCTAATGCTCACCTTTATCACCTCATTCTGAACCCTTTCGCTGGCT |
| Human | 2488 | TCAAGAGTCTAAACCCCAGATTCTTCAGCTTGATCCTGGAGGCTTTTCTAGTCTGAGCTTCTTTAGCTAGGCTAA |
| Human | 2563 | AACACCTTGGCTTGTTATTGCCTCTACTTTGATTCTTGATAATGCTCACTTGGTCCTACCTATTATCCTTTCTAC |
| Human | 2638 | TTGTCCAGTTCAAATAAGAAATAAGGACAAGCCTAACTTCATAGTAACCTCTCTATTTTAATCAGTTGTTTAATA |
| Human | 2713 | ATTTACAGGTTCTTAGGCTCCATCCTGTTTGTATGAAATTATAATCTGTGGATTGGCCTTTAAGCCTGCATTCTT |
| Human | 2788 | AACAAACTCTTCAGTTAATTCTTAGATACACTAAAAATCTGAAGAAACTCTACATGTAACTATTTCTTCAGAGTT |
| Human | 2863 | TGTCATATACTGCTTGTCATCTGCATGTCTACTCAGCATTTGATTAACATTTGTGTAATAAGAAATAAAATTACA |
| Human | 2938 | CAGTAAGTCATTTAACCAAAAAAAAAAAAAAAAAAAAAAAAAAAAAAAAAAAAAAAAAAAAAAAAAAA |

TABLE 3

| Human | − | MSFPKAPLKRENDPSGCAPSPGAYDVKTLEVLKGPVSFQKSQRFKQQKESKQNLNVDKDTTLPASARKVKSSESK |
|---|---|---|
| Human | 76 | KESQKNDKDLKILEKEIRVLLQERGAQDRRIQDLETELEKMEARLNAALREKTSLSANNATLEKQLIELTRTNEL |
| Human | 151 | LKSKESENGNQKNLRILSLELMKLRNKRETKMRGMMAKQEGMEMKLQVTQRSLEESQGKIAQLEGKLVSIEKEKI |
| | | \|+\|\|\|\|\|\|\|\|\|\|\|\|\|\|\|\|\| \| \|\|\|\|\|\|+\|\|\| \|\|+ \| \| \|\| \|\|\|\|\|\|\|\|\|\|\|\|\|\| |
| Mouse | 1 | MRALSLELMKLRNKRETKMRSMMVKQEGMELKLQATQKDLTESKGKIVQLEGKLVSIEKEKI |
| Human | 226 | DEKSETEKLLEYIEEISCASDQVEKYKLDIAQLEENLKEKNDEILSLKQSLEENIVILSKQVEDLNVKCQLLEKE |
| | | \|\|\|+\|\|\|\|\|\|\|\|\| \|\|\|\|\|\|\|\|\|\|\|+ +\|\|\|\|\|\|\| \|\|\|\| \|\|\|\|\|\|\|\|\|\|\|\|\| + \|\|\| \|\|\| \|\|\|\|\|\|\| |
| Mouse | 63 | DEKCETEKLLEYIQEISCASDQVEKCKVDIAQLEEDLKEKDREILSLKQSLEENITF-SKQIEDLTVKCQLLETE |
| Human | 301 | KEDHVNRNREHNENLNAEMQNLKQKFILEQQEHEKLQQKELQIDSLLQQEKELSSSLHQKLCSFQEEMVKEKNLF |
| | | ++ \|++ \|\|+ \|+ +\|\|\|\| \| ++++\|\|+\|\| \|\|\|\|\|\|\|\|\| \|\|\|\|\|\|\|\|\|\|+ \| \|+\|\|\|\|\|\|\|\| \|\|\|+\| |
| Mouse | 138 | RDNLVSKDRERAETLSAEMQILTERLALERQEYEKLQQKELQSQSLLQQEKELSARLQQQLCSFQEEMTSEKNVF |
| Human | 376 | EEELKQTLDELDKLQQKEEQAERLVKQLEEEAKSRAEELKLLEEKLKGKEAELEKSSAAHTQATLLL-------- |
| | | \|\|\|\| \| \|\|\| +\|\|\|\|\|\|+\|\|\|\|\|\|\|\|\|\| \|\| \|\|+\| \|+ \|+ \|\| \|\|\|\| \|\|\| \|\| + |
| Mouse | 213 | KEELKLALAELDAVQQKEEQSERLVKQLEEERKSTAEQLTRLDNLLREKEVELEKHIAAHAQAILIAQEKYNDTA |
| Human | | --------------------------------------------------------------------------- |
| Mouse | 288 | QSLRDVTAQLESVQEKYNDTAQSLRDVTAQLESEQEKYNDTAQSLRDVTAQLESEQEKYNDTAQSLRDVTAQLES |
| Human | 443 | QEKYDSMVQSlEDVTAQFESYKALTASEIEDLKLENSSLQEKAAKAGKNAEDVQHQILATESSNQEYVRMLLDLQ |
| | | \|\|\|\|+ \|\| \|\|\|\|\| \|\|\|\|+ \| \|\|\|\|\|\|\|\|\| +\|\|\|\| \| \| \|+ \|\|\|\| \|\| \| +\|\|\|\| \|\|+ \|\|\| |
| Mouse | 363 | QEKYNDTAQSLRDVTAQLESYKSSTLKEIEDLKLENLTLQEKVAMAEKSVEDVQQQILTAESTNQEYARMVQDLQ |
| Human: | 518 | TKSALKETEIKEITVSFLQKITDLQNQLKQQEEDFRKQLEDEEGRKAEKENTTAELTEEINKWRLLYEELYNKTK |
| | | +\| \|\|\| \|\|\|\|\|\| \|\|\|+\|\|\|\|\|+\|\|\|+\|\|+\|\|\|\|\|\|\|\|++ \| \|\|\|\|\| \|\|\| \|\|\|\|\|\|\|\|\|\|\|\|\| \|\|\| |
| Mouse: | 438 | NRSTLKEEEIKEITSSFLEKITDLKNQLRQQDEDFRKQLEEKGKRTAEKENVMTELTMEINKWRLLYEELYEKTK |
| Human | 593 | PFQLQLDAFEVEKQALLNEHGAAQEQLNKIRDSYAKLLGHQNLKGKIKAVVKLKDENSQLKSEVSKLRCQLAKKK |
| | | \|\|\| \|\|\|\|\|\| \|\|\|\|\|\|\|\|\|\|\| \|\|\|\|\|\|\|\|\|\|\|\|+\|\|\|\|\|\|\|\|\|\|\|\|\|\|\|\|\|\|\|\|\|\|\|\|\|\|\|\|\|\|\|\|+\|\| \|+\| |
| Mouse: | 513 | PFQQQLDAFEAEKQALLNEHGATQEQLNKIRDSYAQLLGHQNLKGKIKHVVKLKDENSQLKSEVSKLRSQLVKRK |
| Human: | 668 | QAETKLQEELNKVLGIKHFDPSKAFHHESKENFALKTPLKEGNTNCYRAPMECQESWK* |
| | | \|+\|++\|\| \|\|+\| \|\|\|+\|\|\|\|\|\|\|\| \| \|\|\|\|\| \|\|\|\|\|\| \|\| |
| Mouse: | 588 | QNELRLQGELDKALGIRHFDPSKAFCHASKENF---TPLKEGNPNCC* |

TABLE 4
Distribution of Staining Scores Among 400 Breast Tumors

| Staining | | General | | Maximum |
|---|---|---|---|---|
| Score | Stromal | Tumor | Nuclear | Tumor |
| 0.0 | 331 | 21 | 323 | 3 |
| 0.5 | 35 | 74 | 16 | 24 |
| 1.0 | 18 | 73 | 10 | 28 |
| 1.5 | 8 | 92 | 5 | 54 |
| 2.0 | 3 | 86 | 18 | 69 |
| 2.5 | 4 | 34 | 2 | 70 |
| 3.0 | 1 | 17 | 14 | 72 |
| 3.5 | 0 | 3 | 4 | 54 |
| 4.0 | 0 | 0 | 8 | 26 |

TABLE 5
Univariate analysis of prognostic indicators for
metastasis-free and overall survival

| | Metastasis-Free Survival | | | Overll Survival | | |
|---|---|---|---|---|---|---|
| | Num- | | | Num- | | |
| Factor | ber | 5-yr Surv | p-value | ber | 5-yr Surv | p-value |
| Nodal status | | | | | | |
| None positive | 179 | 83% | | 186 | 80% | |
| >1 positive | 162 | 50% | <0.0001 | 179 | 60% | <0.0001 |
| Tumor size | | | | | | |
| (cm) | | | | | | |
| 2 | 181 | 70% | | 199 | 72% | |
| 2.01-5 | 149 | 69% | | 160 | 69% | |
| >5 | 22 | 40% | 0.03 | 26 | 58% | 0.01 |
| Tumor grade | | | | | | |
| 1 or 2 | 292 | 70% | | 235 | 72% | |
| 3 | 71 | 62% | 0.74 | 155 | 64% | 0.53 |
| ER status | | | | | | |
| Negative | 23 | 74% | | 24 | 70% | |
| Positive | 173 | 74% | 0.75 | 187 | 78% | 0.67 |
| Age | | | | | | |
| <50 | 96 | 65% | | 103 | 68% | |
| 50 | 265 | 65% | 0.75 | 293 | 68% | 0.19 |
| Stromal stain | | | | | | |
| None | 301 | 70% | | 329 | 70% | |
| Some | 62 | 54% | 0.20 | 69 | 28% | 0.13 |
| General stain | | | | | | |
| 0-0.5 | 95 | 70% | | 95 | 72% | |
| 1-1.5 | 152 | 58% | | 165 | 62% | |
| >2 | 126 | 68% | 0.17 | 138 | 70% | 0.15 |
| Nuclear stain | | | | | | |
| None | 292 | 72% | | 322 | 68% | |
| Some | 71 | 62% 0.74 | 76 | 68% | 0.64 | |

TABLE 6
Multivariate analysis of prognostic factors for
metastasis-free and overall survival

| | Metastasis-Free Survival | | Overall Survival | |
|---|---|---|---|---|
| Factor | Odds Ratio | p. value | Odds Ratio | P. value |
| Nodal status | 2.96¹ | <0.0001 | 2.14¹ | <0.0001 |
| Max-Gen RHAMM | 1.40² | <0.016 | 1.59² | <0.008 |
| Tumor Size | | <0.01 | | <0.004 |
| 2-5 cm | 1.25³ | <0.02 | 1.46³ | <0.08 |
| >5 cm | 1.99³ | <0.003 | 1.61³ | <0.002 |

¹Odds ratios for ≧ vs. 0 positive nodes
²Odds ratios for Max-Gen staining ≧ vs. ≦1
³Odds ratios for tumor size shown vs. Tumor size ≦2 cm

TABLE 7
RHAMM mRNA expression and tumor grade

| RHAMM isoform | Case Number | Median Grade | p value* |
|---|---|---|---|
| RHAMMv4 | | | |
| −/+ | 44 | 6 | |
| ++ | 54 | 7 | 0.0466 |
| RHAMMv4 (−9) | | | |
| − | 70 | 7 | |
| + | 28 | 8 | 0.0163 |
| Both isoforms | | | |
| −/+ | 40 | 6 | |
| ++/+++ | 54 | 7 | 0.0357 |

*Mann Whitney Test.

TABLE 8
RHAMM mRNA expression and prognostic parameters*

| RHAMM | Poor Prognosis | Good Prognosis | |
|---|---|---|---|
| isoform | (12 cases) | (15 cases) | p value** |
| RHAMMv4 | | | |
| −/+ | 2 (17%) | 11 (73%) | |
| ++ | 10 (83%) | 4 (27%) | 0.0063 |
| RHAMMv4 | | | |
| (−9) | | | |
| − | 5 (42%) | 14 (93%) | |
| + | 7 (58%) | 1 (7%) | 0.0085 |
| Both isoforms | | | |
| −/+ | 3 (25%) | 11 (73%) | |
| ++/+++ | 9 (75%) | 4 (27%) | 0.0213 |

*Poor prognosis parameters: high grade/ER − ve/Node + ve. Good prognosis parameters: low grade/ER + ve/Node − ve.
**Fisher Exact Test.

We claim:

1. An isolated nucleic acid comprising a nucleotide sequence encoding a protein selected from the group consisting of human RHAMM 1, human RHAMM 2, human RHAMM 3, human RHAMM 4 and human RHAMM 5.

2. The isolated nucleic acid of claim 1 wherein the nucleic acid encodes the amino acid sequence of Sequence ID NO:4.

3. The isolated nucleic acid of claim 2 wherein the nucleotide sequence is selected from the group consisting of
(a) the genomic sequence of human RHAMM; and
(b) the nucleotide sequence of Sequence ID NO:3.

4. The isolated nucleic acid of claim 1 selected from the group consisting of
(a) a nucleotide sequence comprising in continuous sequence the nucleotide sequences of Sequence ID NOS:9 to 25;
(b) a nucleotide sequence comprising in continuous sequence the nucleotide sequences of Sequence ID NOS:9, 10, 11 and 13 to 25; and
(c) a nucleotide sequence comprising in continuous sequence the nucleotide sequences of Sequence ID NOS:9, 10 and 12 to 25.

5. An isolated nucleic acid comprising a nucleotide sequence selected from the group consisting of
(a) a nucleotide sequence of at least 10 consecutive nucleotides of Sequence ID NO:3;
(b) a nucleotide sequence of at least 15 consecutive nucleotides of Sequence ID NO:3; and
(c) a nucleotide sequence of at least 20 consecutive nucleotides of Sequence ID NO:3.

6. An isolated nucleic acid comprising a nucleotide encoding at least one binding domain of human RHAMM protein or a fragment or analogue thereof which retains HA binding ability.

7. The isolated nucleic acid of claim 6 encoding the amino acid sequence of Sequence ID NO:1 or Sequence ID NO:7.

8. An isolated nucleic acid comprising a nucleotide sequence selected from the group consisting of Sequence ID NO:9, Sequence ID NO:10, Sequence ID NO:11, Sequence ID NO:12, Sequence ID NO:13, Sequence ID NO:14, Sequence ID NO:15, Sequence ID NO:16, Sequence ID NO:17, Sequence ID NO:18, Sequence ID NO:19, Sequence ID NO:20, Sequence ID NO:21, Sequence ID NO:22, Sequence ID NO:23, Sequence ID NO:24 and Sequence ID NO:25.

9. The nucleic acid of claim 8 wherein the nucleotide sequence is Sequence ID NO:16.

10. An isolated nucleic acid comprising a nucleotide sequence encoding the amino acid sequence of Sequence ID NO:50.

11. An isolated nucleic acid comprising a nucleotide sequence selected from the group consisting of the Sequences ID NO:26 to 49.

12. A recombinant expression vector comprising an isolated nucleic acid of any of claims 1 to 11.

13. A host cell transformed with a recombinant expression vector of claim 12.

14. A transgenic animal wherein a genome of the animal, or of an ancestor thereof, has been modified by insertion of at least one recombinant construct to produce a modification selected from the group consisting of
(a) insertion of a nucleotide sequence of at least one exon of the human RHAMM gene;
(b) insertion of a nucleotide sequence encoding at least one human RHAMM protein;
(c) inactivation of an endogenous RHAMM gene.

15. A substantially pure protein selected from the group consisting of human RHAMM 1, human RHAMM 2, human RHAMM 3, human RHAMM 4 and human RHAMM 5.

16. The protein of claim 15 comprising the amino acid sequence of Sequence ID NO:4 or a fragment or analogue thereof which retains the ability to bind hyaluronan.

17. A substantially pure peptide comprising an amino acid sequence selected from the group consisting of
(a) at least 5 consecutive amino acid residues from the amino acid sequence of Sequence ID NO:4;
(b) at least 10 consecutive amino acid residues from the amino acid sequence of Sequence ID NO:4; and
(c) at least 15 consecutive amino acid residues from the amino acid sequence of Sequence ID NO:4.

18. A substantially pure peptide comprising at least one binding domain of human RHAMM.

19. A peptide of claim 18 selected from the group consisting of Sequence ID NO:1 and Sequence ID NO:17.

20. A substantially pure peptide having the amino acid sequence of Sequence ID NO:50.

21. An antibody which selectively binds to an antigenic determinant of a human RHAMM protein.

22. An antibody which selectively binds to an antigenic determinant of the peptide of claim 20.

23. A cell line producing an antibody of claim 21 or 22.

24. A method for identifying compounds which can selectively bind to a human RHAMM protein comprising the steps of
providing a preparation of at least one human RHAMM protein;
contacting the preparation with a candidate compound; and
detecting binding of the RHAMM protein to the candidate compound.

25. The method of claim 24 wherein the binding of the RHAMM protein to the compound is detected by a method selected from the group consisting of affinity chromatography, a yeast two-hybrid system, and a phage display library.

26. A method for assessing prognosis in a mammal having a tumour, comprising obtaining a tumour sample from the mammal and determining the level of expression of RHAMM protein in the tumour sample, wherein increased expression of RHAMM protein is indicative of a poor prognosis.

27. The method of claim 26 wherein RHAMM expression is determined by a method selected from the group consisting of a histochemical method, a method comprising determination of the level of RHAMM mRNA in a biopsy sample and a method comprising determination of expression of human RHAMM exon 8 in a biopsy sample.

28. The method of any of claims 26 to 27 wherein the mammal is a human and the tumour is a breast tumour.

29. A pharmaceutical composition for preventing or treating a disorder in a human characterised by overexpression of the RHAMM gene comprising an effective amount of a nucleotide sequence selected from the group consisting of
(a) a dominant suppressor mutant of the RHAMM gene;
(b) an antisense sequence to human RHAMM cDNA; and
(c) an antisense sequence to exon 8 of the human RHAMM gene and a pharmaceutically acceptable carrier.

30. A method for preventing or treating a disorder in a human characterised by overexpression of the RHAMM gene comprising administering to the mammal an effective amount of a nucleotide sequence selected from the group consisting of
(a) a dominant suppressor mutant of the RHAMM gene;
(b) an antisense sequence to human RHAMM cDNA; and
(c) an antisense sequence to exon 8 of the human RHAMM gene.

31. the method of claim 32 wherein the disorder is cancer.

32. A method for inhibiting cell migration in a human comprising administering to the human an effective amount of an agent selected from the group consisting of
(a) an antibody which binds specifically to human RHAMM protein or a fragment thereof; and
(b) a peptide comprising a human RHAMM HA-binding domain.


SEQUENCE LISTING (52 sequences)

SEQ ID NO: 1 - protein, 11 aa
KQKIKHVVKLK

SEQ ID NO: 2 - protein, 10 aa
KLRSQLVKRK

SEQ ID NO: 3 - DNA (cDNA), 3114 bp; single-stranded, linear
ccgccagtgtgatggatatctgcagaattcggcttactcactatagggctcgagcggccgcccgggcaggtgtgccagtcaccttcagtttctggagctggccgtcaacatgtcctttcctaaggcgcccttgaaacgattcaatgacccttctggttgtgcaccatctccaggtgcttatgatgttaaaactttagaagtattgaaaggaccagtatcctttcagaaatcacaaagatttaaacaacaaaaagaatctaaacaaaatcttaatgttgacaaagatactaccttgcctgcttcagctagaaaagttaagtcttcggaatcaaagaaggaatctcaaaagaatgataaagatttgaagatattagagaaagagattcgtgttcttctacaggaacgtggtgcccaggacaggcggatccaggatctggaaactgagttggaaaagatggaagcaaggctaaatgctgcactaagggaaaaaacatctctctctgcaaataatgctacactggaaaaacaacttattgaattgaccaggactaatgaactactaaaatctaagttttctgaaaatggtaaccagaagaatttgagaattctaagcttggagttgatgaaacttagaaacaaaagagaaacaaagatgaggggtatgatggctaagcaagaaggcatggagatgaagctgcaggtcacccaaaggagtctcgaagagtctcaagggaaaatagcccaactggagggaaaacttgtttcaatagagaaagaaaagattgatgaaaaatctgaaacagaaaaactcttggaatacatcgaagaaattagttgtgcttcagatcaagtggaaaaatacaagctagatattgcccagttagaagaaaatttgaaagagaagaatgatgaaattttaagccttaagcagtctcttgaggaaaatattgttatattatctaaacaagtagaagatctaaatgtgaaatgtcagctgcttgaaaaagaaaaagaagaccatgtcaacaggaatagagaacacaacgaaaatctaaatgcagagatgcaaaacttaaaacagaagtttattcttgaacaacaggaacatgaaaagcttcaacaaaaagaattacaaattgattcacttctgcaacaagagaaagaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagggaaggaggctgaactggagaaaagtagtgctgctcatacccaggccaccctgcttttgcaggaaaagtatgacagtatggtgcaaagccttgaagatgttactgctcaatttgaaagctataaagcgttaacagccagtgagatagaagatcttaagctggagaactcatcattacaggaaaaagcggccaaggctgggaaaaatgcagaggatgttcagcatcagattttggcaactgagagctcaaatcaagaatatgtaaggatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaagaaaagctgaaaaagaaaatacaacagcagaattaactgaagaaattaacaagtggcgtctcctctatgaagaactatataataaaacaaaaccttttcagctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcggaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttcgctggctttccagcttagaatgcatctcatcaacttaaaagtcagtatcatattattatcctcctgttctgaaaccttagtttcaagagtctaaaccccagattcttcagcttgatcctggaggcttttctagtctgagcttctttagctaggctaaaacaccttggcttgttattgcctctactttgattcttgataatgctcacttggtcctacctattatcctttctacttgtccagttcaaataagaaataaggacaagcctaacttcatagtaacctctctattttaatcagttgtttaataatttacaggttcttaggctccatcctgtttgtatgaaattataatctgtggattggcctttaagcctgcattcttaacaaactcttcagttaattcttagatacactaaaaatctgaagaaactctacatgtaactatttcttcagagtttgtcatatactgcttgtcatctgcatgtctactcagcatttgattaacatttgtgtaataagaaataaaattacacagtaagtcatttaaccaaaaaaaaaaaaaaaaaaaaaaaaaaaaaaaaaaaaaaaaaaaaaaaaaaa

SEQ ID NO: 4 - protein, 725 aa [sequence omitted]

SEQ ID NO: 5 - DNA (cDNA), 2123 bp; single-stranded, linear
aggctaaaggaggcagaatagatatctgagttcttatgtttattgtagttttctgaagatggtcaccaaaagaatatgagagctctaagcctggaattgatgaaactcagaaataagagagagacaaagatgaggagtatgatggtcaaacaggaaggcatggagctgaagctgcaggccactcagaaggacctcacggagtctaagggaaaaatagtccagctggagggaaagcttgtttcaatagagaaagaaaagatcgatgaaaaatgtgaaacagaaaaactcttagaatacatccaagaaattagctgtgcatctgatcaagtggaaaaatgcaaagtagatattgcccagttagaagaagatttgaaagagaaggatcgtgagattttaagtcttaagcagtctcttgaggaaaacattacattttctaagcaaatagaagacctgactgttaaatgccagctacttgaaacagaaagagacaaccttgtcagcaaggatagagaaagggctgaaactctcagtgctgagatgcagatcctgacagagaggctggctctggaaaggcaagaatatgaaaagctgcaacaaaaagaattgcaaagccagtcacttctgcagcaagagaaggaactgtctgctcgtctgcagcagcagctctgctctttccaagaggaaatgacttctgagaagaacgtctttaaagaagagctaaagctcgccctggctgagttggatgcggtccagcagaaggaggagcagagtgaaaggctggttaaacagctggaagaggaaaggaagtcaactgcagaacaactgacgcggctggacaacctgctgagagagaaagaagttgaactggagaaacatattgctgctcacgcccaagccatcttgattgcacaagagaagtataatgacacagcacagagtctgagggacgtcactgctcagttggaaagtgtgcaagagaagtataatgacacagcacagagtctgagggacgtcactgctcagttggaaagtgagcaagagaagtacaatgacacagcacagagtctgagggacgtcactgctcagttggaaagtgagcaagagaagtacaatgacacagcacagagtctgagggacgtcactgctcagttggaaagtgtgcaagagaagtacaatgacacagcacagagtctgagggacgtcagtgctcagttggaaagctataagtcatcaacacttaaagaaatagaagatcttaaactggagaatttgactctacaagaaaaagtagctatggctgaaaaaagtgtagaagatgttcaacagcagatattgacagctgagagcacaaatcaagaatatgcaaggatggttcaagatttgcagaacagatcaaccttaaaagaagaagaaattaaagaaatcacatcttcatttcttgagaaaataactgatttgaaaaatcaactcagacaacaagatgaagactttaggaagcagctggaagagaaaggaaaaagaacagcagagaaagaaaatgtaatgacagaattaaccatggaaattaataaatggcgtctcctatatgaagaactatatgaaaaaactaaaccttttcagcaacaactggatgcctttgaagccgagaaacaggcattgttgaatgaacatggtgcaactcaggagcagctaaataaaatcagagactcctatgcacagctacttggtcaccagaacctaaagcaaaaaatcaaacatgttgtgaaattgaaagatgaaaatagccaactcaaatcggaggtgtcaaaactccgatctcagcttgttaaaaggaaacaaaatgagctcagacttcagggagaattagataaagctctgggcatcagacactttgacccttccaaggctttttgtcatgcatctaaggagaattttactccattaaaagaaggcaacccaaactgctgctgagttcagatgcaacttcaagaatcatggaagtatacgtctgaaatacttgttgaagattattttcttcattgttcttgttatagtatataatgtatttaatttctactgcctagtcttaggtatatgaaacggtaattcagcatttgttctct

SEQ ID NO: 6 - protein, 630 aa
MRALSLELMKLRNKRETKMRSMMVKQEGMELKLQATQKDLTESKGKIVQLEGKLVSIEKEKIDEKCETEKLLEYIQEISCASDQVEKCKVDIAQLEEDLKEKDREILSLKQSLEENITFSKQIEDLTVKCQLLETERDNLVSKDRERAETLSAEMQILTERLALERQEYEKLQQKELQSQSLLQQEKELSARLQQQLCSFQEEMTSEKNVFKEELKLALAELDAVQQKEEQSERLVKQLEEERKSTAEQLTRLDNLLREKEVELEKHIAAHAQAILIAQEKYNDTAQSLRDVTAQLESVQEKYNDTAQSLRDVTAQLESEQEKYNDTAQSLRDVTAQLESEQEKYNDTAQSLRDVTAQLESQEKYNDTAQSLRDVTAQLESYKSSTLKEIEDLKLENLTLQEKVAMAEKSVEDVQQQILTAESTNQEYARMVQDLQNRSTLKEEEIKEITSSFLEKITDLKNQLRQQDEDFRKQLEEKGKRTAEKENVMTELTMEINKWRLLYEELYEKTKPFQQQLDAFEAEKQALLNEHGATQEQLNKIRDSYAQLLGHQNLKQKIKHVVKLKDENSQLKSEVSKLRSQLVKRKQNELRLQGELDKALGIRHFDPSKAFCHASKENFTPLKEGNPNCC

SEQ ID NO: 7 - protein, 10 aa
KLRCQLAKKK

SEQ ID NO: 8 - protein, 10 aa
KLRSQLAKRK

SEQ ID NO: 9 - DNA, 155 bp; single-stranded, linear
ccgccagtgtgatggatatctgcagaattcggcttactcactatagggctcgagcggccgcccgggcaggtgtgccagtcaccttcagtttctggagctggccgtcaacatgtcctttcctaaggcgcccttgaaacgattcaatgacccttctg

SEQ ID NO: 10 - DNA, 99 bp; single-stranded, linear
gttgtgcaccatctccaggtgcttatgatgttaaaactttagaagtattgaaaggaccagtatcctttcagaaatcacaaagatttaaacaacaaaaag

SEQ ID NO: 11 - DNA, 78 bp; single-stranded, linear
aatctaaacaaaatcttaatgttgacaaagatactaccttgcctgcttcagctagaaaagttaagtcttcggaatcaa

SEQ ID NO: 12 - DNA, 122 bp; single-stranded, linear
agaaggaatctcaaaagaatgataaagatttgaagatattagagaaagagattcgtgttcttctacaggaacgtggtgcccaggacaggcggatccaggatctggaaactgagttggaaaag

SEQ ID NO: 13 - DNA, 117 bp; single-stranded, linear
atggaagcaaggctaaatgctgcactaagggaaaaaacatctctctctgcaaataatgctacactggaaaaacaacttattgaattgaccaggactaatgaactactaaaatctaag

SEQ ID NO: 14 - DNA, 87 bp; single-stranded, linear
ttttctgaaaatggtaaccagaagaatttgagaattctaagcttggagttgatgaaacttagaaacaaaagagaaacaaagatgagg

SEQ ID NO: 15 - DNA, 101 bp; single-stranded, linear
ggtatgatggctaagcaagaaggcatggagatgaagctgcaggtcacccaaaggagtctcgaagagtctcaagggaaaatagcccaactggagggaaaact

SEQ ID NO: 16 - DNA, 75 bp; single-stranded, linear
tgtttcaatagagaaagaaaagattgatgaaaaatctgaaacagaaaaactcttggaatacatcgaagaaattag

SEQ ID NO: 17 - DNA, 179 bp; single-stranded, linear
ttgtgcttcagatcaagtggaaaaatacaagctagatattgcccagttagaagaaaatttgaaagagaagaatgatgaaattttaagccttaagcagtctcttgaggaaaatattgttatattatctaaacaagtagaagatctaaatgtgaaatgtcagctgcttgaaaaagaaaaag

SEQ ID NO: 18 - DNA, 149 bp; single-stranded, linear
aagaccatgtcaacaggaatagagaacacaacgaaaatctaaatgcagagatgcaaaacttaaaacagaagtttattcttgaacaacaggaacatgaaaagcttcaacaaaaagaattacaaattgattcacttctgcaacaagagaaa

SEQ ID NO: 19 - DNA, 215 bp; single-stranded, linear
gaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagg

SEQ ID NO: 20 - DNA, 117 bp; single-stranded, linear
gaaggaggctgaactggagaaaagtagtgctgctcatacccaggccaccctgcttttgcaggaaaagtatgacagtatggtgcaaagccttgaagatgttactgctcaatttgaaag

SEQ ID NO: 21 - DNA, 147 bp; single-stranded, linear
ctataaagcgttaacagccagtgagatagaagatcttaagctggagaactcatcattacaggaaaaagcggccaaggctgggaaaaatgcagaggatgttcagcatcagattttggcaactgagagctcaaatcaagaatatgtaag

SEQ ID NO: 22 - DNA, 153 bp; single-stranded, linear
gatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaag

SEQ ID NO: 23 - DNA, 100 bp; single-stranded, linear
aaaagctgaaaaagaaaatacaacagcagaattaactgaagaaattaacaagtggcgtctcctctatgaagaactatataataaaacaaaaccttttcag

SEQ ID NO: 24 - DNA, 177 bp; single-stranded, linear
ctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcg

SEQ ID NO: 25 - DNA, 1040 bp; single-stranded, linear
gaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttgctggctttccagcttagaatgcatctcatcaacttaaaagtcagtatcatattattatcctcctgttctgaaaccttagtttcaagagtctaaaccccagattcttcagcttgatcctggaggtcttttctagtctgagcttctttagctaggctaaaacaccttggcttgttattgcctctactttgattctgataatgctcacttggtcctacctattatccttctacttgtccagttcaataagaaataaggacaagcctaacttcatagtaacctctctattttaatcagttgtttaataatttacaggttcttaggctccatcctgtttgtatgaaattataatctgtggattggcctttaagcctgcattcttaacaaactcttcagttaattcttagatacactaaaaatctgaagaaactctacatgtaactatttcttcagagtttgtcatatactgcttgtcatctgcatgtctactcagcatttgattaacatttgtgtaataagaaataaaattacacagtaagtcatttaaccaaaaaaaaaaaaaaaaaaaaaaaaaaaaaaaaaaaaaaaaaaaaaaaaaaa

SEQ ID NO: 26 - DNA (genomic), 264 bp; single-stranded, linear
gtgcgtaagggggaaagagctgggggggggagacgccctaacgccctttgcctctttcagctcccttcttgggaggcaagcaggaggcgattttagggtcgggctggggctcattcagttgattgatttttctcaaatatgctctaagcatctgttacatgccaagcactaatcaggatgctaaggataccgcagtaaacagtctccgcccgtgggcttacattcaggcggggaatactgtcaataaacagcggtaatggagaa

SEQ ID NO: 27 - DNA (genomic), 291 bp; single-stranded, linear
ttcaatccttagtaagaaagccatatattgcctgaatatatgatgtcatctcaaaactgcgtttgctcagttgcctgtgttcctttgacccggttgatataaagggcaagatgatattgttcttcatagagaggccttctttgtaatatcaaatggatgcaattttttacatttaaaaaaagcagtttgttaatgacatttttacatttatattcactttattatgacatgttttaacttaagatcataagtaacattagataatatattaatgttttctatttcctctag

SEQ ID NO: 28 - DNA (genomic), 227 bp; single-stranded, linear
gtaataagatcaccaaagaacaatggttatgtgatcttataagttttaaagttatgaataacaatatttaaagatgttatagcattttttaaaatgtgaagctagaactatatttaaattttatttgatggatttatgaaagggtcaagtacagaataatgctgtcatcattacattgttatataaccaggaaaattaagcaagatacttatattgatatgtagctt

SEQ ID NO: 29 - DNA (genomic), 244 bp; single-stranded, linear
ggtgaggagcttttatatgccagctggtttatcaagtgtatcatcaaaaacatctgaaagtattgtatttgattagaatgggttaaagtgtatgaatcaaggttataactaaatctgtaaattaatgaaatgagttatcattagaactctagcaagttttacatttctgcctaggtcattatgtttaaatgtgcccttagttcacaattataatggtcttcaattctcaatcacttctatgttt

SEQ ID NO: 30 - DNA (genomic), 274 bp; single-stranded, linear
ttatgttcttaaaatgcattaagactttaagatgtatcataggtaaatatgattattcaaatagctagtaacattagaatatctacaagcataatgtcaaaatcagagatttttccagaaactttaggggtgattattggtagcatctccttatgttggcattctatcagtgaatcatttattatcaccttgtttttgtccagattcgtgttcttctacaggaacgtggtgcccaggacaggcggatccaggatctggaaactgagttggaaag

SEQ ID NO: 31 - DNA (genomic), 286 bp; single-stranded, linear
gtatctgagcctcatgataacatttacaattgaataaatataaacacgttttttagggccgggcacggtggctcacgcctgtgttcccagcattttgggaggccaaggcaggcggatcacctgaggtcgggagttcgagaccagcctgaccaacatggagaaaccctgtctctactaaaaatacaaaaattagctaggcctattggcgggcgcctgtaatctcagctactcgggaggctgaggcagaagaatcacttgaacccaggaggtggaggttgcagtgagc

SEQ ID NO: 32 - DNA (genomic), 521 bp; single-stranded, linear
taaaccagcaagtcacattaaggaaaagagggataagaacagtagactggtacagtggctcatgcctgtatttccagcattttggaaggctgagggctggagaattgcttgaggccaggagtttgagaccagcctgggcaacatatcaagaccccatctctataaacaaattgaaaaattagctaggcatggtggtggtgcacaccggtaatcccagctactcaggaagatgaggcaggaggattgattgagcccaggagtttgagattatagcgagctatgatcatgccactccactctagccgtgacagcggagcgagacttgatctcttaaaaagaaaagaaaaaaaaattaaatcaatcagtaattatggtgtaggtcaaagactgttctctctaccaaagtatattaaagtcaaaaacataaccccagtgataggtagaaaaatcaatatttctctattttaaatatgtcttagcagaaaatatttctgaattttttacgtgtttgttgtatttag

SEQ ID NO: 33 - DNA (genomic), 91 bp; single-stranded, linear
gtgagtgctgcccttggcaggtttgctgtgtctggatctggggatcagtacaactttctcatttcctaaaacaggtatctttgttgtgtag

SEQ ID NO: 34 - DNA (genomic), 467 bp; single-stranded, linear
gtaagtgagtgaatgtgaagagaaattgttaagtggaagcaattcttgatttgagtctcttcacaattattgtttactagacttaaccttctcttagtacttatctcattgcctccctccagttgccctatttctctttttaaactagaatgagccctaatcattctcaaacatgttgtgctacaaagttgtatgagtgcattacttttgtacatcttctgtattattaatgatgaggaaagatttcatgatcttatgaaagtggtcattagattgaaattgagaaacactggtataggaaattgtgatttatgcacaatcctagcctttgattttgagctttaatatacatataataaaatgtgtggatagtaagtattcagtttggtgactttagcaattgtatacacctactaaccactaccaaacaagatagaacattttcatcccttcagaaagttccttca

SEQ ID NO: 35 - DNA (genomic), 549 bp; single-stranded, linear
ttctactaggtaggaagtggtatctcctttgtgattttaatttgttaccatgaatgttgaccttatttttatgtgcttattgaccattttatgtgcatacaacttttgcaaggtgtctattgaagtcttttgtccatttcttgcattggacagtttggtggaggtaaacagataagtaattgaagaccaggtagtctgggacaaaagctttatgggcacacaaaatgctatttagtatgttggatgggtggggaaaccaggaagaccacaaaaagaatattatttctaacacttgggatactgtaatgaaggttctgtcatcataggtttttttgcagtatatattcagaaaactttctcacttaaataaaaattttagtcttctattttgatgtaaattgtgatttgagaaattacataaaataatagttaagagttagggctctgtagtcagcctgcctgatacaggagtatctggtacataagcattatgtaagattattaaataacgaaactagaatgtattaacatatgcaatttttgttttag

SEQ ID NO: 36 - DNA (genomic), 125 bp; single-stranded, linear
gtaatatgagcagtagctttaaattgaaccttatttttttaatactcagtcattttcatcatttttctgttattttccctgtgcctaaatagatgtgctttttaagataatttgttttaatgcag

SEQ ID NO: 37 - DNA (genomic), 497 bp; single-stranded, linear
gtattacagtgtttatagttactttgtttagataagtgttacatacaacatttaggaaaaatactactatgctaaaacaaccttttaaatataattagctatactaacattttaaatataattagctatatagctatacaacagcaaaaacctgtactgcattttagaatattttactcttataatgtttgttttctgtttatttcaatacagcatattacctgtcttgattgaaatatatacagtcatataattcttgactttccactaggtagctgtgtaacaatcagtagataacacagaacaagatttgtgggttttattatttagcacatagtatatattacatggagtaatgatacaaagttcacagttttgttttcttctttggaaataccatgctaaaagcagtgtaatggaatattatgggagtccaggtttctcagtcttaatgttcttatctaattccagtattcttgatgttttgagttttctag

SEQ ID NO: 38 - DNA (genomic), 295 bp; single-stranded, linear
gtaatttaccaccatatttttttaaactgttcattttgtgtcatacatttccctatgtctctgaacacctttaaattgtgtatatcctttgatctaccaattctatctttagagtcttatcctgaggacataatcatggatatgctgaggatttagctacgtattttcactacatgttcacctagggttatgaataatgtgggaaatgacaacagatacaaaatagggaatttttaaaaaattttctggctcattcttgtgttatttaggctatataaacattacacttaccttg

SEQ ID NO: 39 - DNA (genomic), 328 bp; single-stranded, linear
taattttatgtaatatggtgtgaaaaataatgttaatatcaaagccagttgtaaaacagatatatatatataaaaatataattttagattaagaagtttctgcatgtgcgttgcatagaaaaaagcctaagatgatatttgccacaatgttaacaaggtataggaaataatctatgaaaacaaatatgctatttctatattgttttaagtttccttgaatctgtggaatttaggtttcatccttctttatctgtacttttttttgtctcctagtacaacctcacaatgccattccaaattattttggtggttttctgtttggatatag

SEQ ID NO: 40 - DNA (genomic), 112 bp; single-stranded, linear
gtttgtattaataggatctcatgttttattatgacttcagatgtatttattttgagtactttttttagtattctcttatcaatcatgtgagcgtgttaggttggattatttt

SEQ ID NO: 41 - DNA (genomic), 255 bp; single-stranded, linear
ttatacctactaccttcttcacccaaatttttaaagtaaaataagcaggaaagataagttgaagctagtagaaaaatgcattaaaaaacatgctttcgaggtaagtcataaattaggatctgagctatttagcaggtaatgcagtggtgaagatatgagctatatgattcacagtttcaaaggtaaatactattttctttcttagggtagtaattgtaggtggcattttatctttcaattatttctttttcttag

SEQ ID NO: 42 - DNA (genomic), 206 bp; single-stranded, linear
gtatttttcttgggagcctgcactcttaaatatgatgtgtgcagaaaggggtgtttaccccaggaaatatgtgagcaaagcagtcacacaaaggatgattcatactagtttaaattccataatcaccaaccgtaagtgggcatttagcattatctggtaatcttattgtatttatataattccctttataatttatagaaattccc

SEQ ID NO: 43 - DNA (genomic), 207 bp; single-stranded, linear
tttttttttctttgaatacacagcagatgccatgtaaactcattagtacttgcctcagaacactgaattcttacctgtgttaaatgcatgaatacattaaaaactttttagttttacttagaagtatataaagtgtcccctaatcagttatgattgtcatacgcaatagttagaaaactactttgacttttttttctttttaataag

SEQ ID NO: 44 - DNA (genomic), 231 bp; single-stranded, linear
gtatatagagcaaataatggccttagaaccattaagacaatttaatgttgaaagccagctagtaactgtcccttggcttgcttttggccatcttatactgcaaattaagaatttactcagttaaaaaatgacacttcttgaagagttccttgaggtttaaagaaaaaaaaaggaaaaattaatgaaagtggctataaaatgtttagtgacctcttctctctcaaaccaaag

SEQ ID NO: 45 - DNA (genomic), 95 bp; single-stranded, linear
gtaatctatgattcgaacctgagtgccttgttaactcagttacgatgtgattttttaaataactatgtttttctcaatttaattcttccatgcag

SEQ ID NO: 46 - DNA (genomic), 249 bp; single-stranded, linear
gtttgtcagttaggagtaaacttacttgtgtttattttagggactctttgttccctattatagtgaggacagtgactcgggttttctgcaagatcattttgctctgcacttacagtgccaatttagctcactattaaaggtttatacattttattaaattatgcataattttttcccacattattgaagtataattgacaaatttaattgacataatttttcaatggacctttgtggttttaaaaaaaa

SEQ ID NO: 47 - DNA (genomic), 151 bp; single-stranded, linear
ctcatagagaatctatggagagccctgagaatatgtgaacataccttgttttcatttgtgtttttaattttctttagtgtttatggtttatatgaaactagtaagatcaaactgttttaagtcttaactttatttaaaaaatctttttcag

SEQ ID NO: 48 - DNA (genomic), 275 bp; single-stranded, linear
gtttgtaaaatgacttttcattttattaaagatattggagtgggggttattctaactataatacttaaataaaatgaatatctttggtatcagaaaaaaataactgtttatagaggaaaattgagctgtgatttagtggatttattttagagtgttgaccagatgggcattcaatgttctaaagttttctagctaccgtcttaatatatattgaaaattacttgagtaaatttgatgaattcattaagctttacatatctatttccatttgcaaa

SEQ ID NO: 49 - DNA (genomic), 282 bp; single-stranded, linear
ggtctgtaggaaaaacgactattgattgggttagcgtcctaatcgagtatgtggttctgtggctgcaacacagatgtccacagtgacaaggacatgaacacctggatgaacgcgtctgtcaagtctgggtgggctgcatcagtgcctttgcctgtcctgtctcttgcctaagccctcctggttctgactgctcctgcctgggtccctccttcacctgaactctgcaggctgcacagacatgctttctgtatctgtggcccttcattgtccctttccgtgtca

SEQ ID NO: 50 - protein, 25 aa
VSIEKEKIDEKSETEKLLEYIEEIS

SEQ ID NO: 51 - DNA (other nucleic acid), 25 bp; single-stranded, linear
  description: /desc = “primer”
ggatatctgcagaattcggcttact

SEQ ID NO: 52 - DNA (other nucleic acid), 24 bp; single-stranded, linear
  description: /desc = “primer”
acagcaacatcaataacaacaaga